(19) United States
(12) Patent Application Publication
(10) Pub. No.: US 2013/0005696 A1
(43) Pub. Date: Jan. 3, 2013

(54) BUFADIENOLIDE DERIVATIVES, PREPARING PROCESS THEREOF, COMPOSITION COMPRISING THE SAME AND THE USE THEREOF

(75) Inventors: Lihong Hu, Shanghai (CN); Dean Guo, Shanghai (CN); Zhiyong Xiao, Shanghai (CN); Xuan Liu, Shanghai (CN); Biao Ma, Shanghai (CN); Min Lei, Shanghai (CN)

(73) Assignee: SHANGHAI INSTITUTE OF MATERIA MEDICA, CHINESE ACADEMY OF SCIENCES, Shanghai (CN)

(21) Appl. No.: 13/508,827

(22) PCT Filed: Feb. 20, 2012
(86) PCT No.: PCT/CN12/71342

§ 371 Date: Jul. 24, 2012

(30) Foreign Application Priority Data
Jun. 30, 2011 (CN) 201110180620.4
Jan. 19, 2012 (CN) 201210017717.8

(51) Int. Cl. A61K 31/585; A61P 35/02; A61P 35/00; C07J 41/00; C07J 43/00

(52) U.S. Cl. 514/171; 540/104; 514/175

(57) ABSTRACT

The present invention provides a class of bufadienolide derivatives representing by the following formula I or pharmaceutically acceptable salts thereof, the preparing process thereof, pharmaceutical composition comprising the same and the use thereof. The bufadienolide derivatives have inhibitory activities against human-derived tumor cell lines, and thus can be used as a drug for treating malignancies.

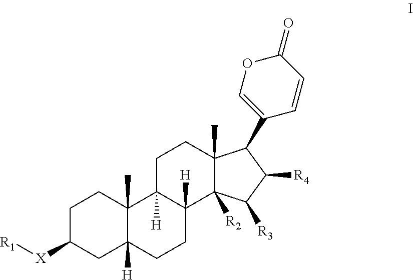

### CROSS REFERENCE OF THE RELATED APPLICATION

This application claims the benefits of the priorities of the Chinese invention patent application No. 201110180620.4 filed with SIPO of China on Jun. 30, 2011 and the Chinese Invention patent application No. 201210017717.8 filed with SIPO of China on Jan. 19, 2012, the disclosures of these applications are incorated herein by reference by their entirety, as totally described herein.

### FIELD OF THE INVENTION

The present invention relates to the field of Pharmaceutical Chemistry, more specifically, to a class of bufadienolide derivatives, the preparing process thereof, pharmaceutical composition comprising the same and the use thereof. The bufadienolide derivatives have inhibitory activities against tumor cell lines, and thus can be used as a drug for treating malignancies.

### BACKGROUND OF THE INVENTION

Tumor is one of the malignant diseases threatening the life of human. More than 5 millions peoples are dying of tumor every year all over the world. In China, more than 1.6 millions tumor patients are newly found and more than 1.3 millions die every year. Therefore, the study on the drugs against the tumor is of especial interest in various countries of the world.

Cardiac glycosides are mainly used for treating cardiac diseases and conditions such as congestive heart failure, arrhythmias, etc. as an inhibitor of sodium pump for a long time. Besides, cardiac glycosides also have the functions of selectively inhibiting the proliferation of tumor cells. Early in 1964, it was reported that cardiac glycosides exhibited strong in vitro activities against nasopharyngeal carcinoma [Kupchan S. M., Hemingway R. J., Doskotch R. W. Tumor inhibitors.IV. Apocannoside and cymarin, the cytotoxic principles of apocynum cannabinum L. J. Med. Chem. 7, 803-804 (1964)]. The subsequent studies confirmed that, cardiac glycosides could induce necrosis of various tumor cells at concentrations lower than the blood drug level used for treating heart failure [Yeh J. Y, Huang W. J., Kan S. F., Wang P. S. Inhibitory effects of digitalis on the proliferation of androgen dependent and independent prostate cancer cells. J. Urology, 166, 1937-1942 (2001); Lopez-Lazaro M., Pastor N., Azrak S. S., Ayuso M. J., Austin C. A., Cortes F. Digitoxin inhibits the growth of cancer cell lines at concentrations commonly found in cardiac patients. J. Nat. Prod. 68, 1642-1645 (2005)]. This important discovery shows that cardiac glycosides might be used as a drug for treating malignancies. Therefore, in the recent 20 years, researchers all over the world focused on studies of cardiac glycosides related to its mechanism of anti-cancer activities, extraction and purification, total synthesis, structural modification, structure-activity relationship, and even clinical trials. Up to now, a large amount of related research articles and reviews have been published [Melero, C. P.; Medarde, M.; Feliciano, A. S., A short review on cardiotonic steroids and their aminoguanidine analogues. Molecules 2000, 5 (1), 51-81.; Chen, J. Q.; Contreras, R. G; Wang, R.; Fernandez, S. V; Shoshani, L.; Russo, I. H.; Cereijido, M.; Russo, J., Sodium/potassium ATPase (Na⁺, K⁺-ATPase) and ouabain/related cardiac glycosides: a new paradigm for development of anti-breast cancer drugs. Breast Cancer Res Tr 2006, 96 (1), 1-15.; Mijatovic, T.; Lefranc, F.; Quaquebeke, E. V.; Vynckt, F. V; Darro, F.; Kiss, R., UNBS1450: A new hemi-synthetic cardenolide with promising anti-cancer activity. Drug Develop Res 2007, 68, 164-173.].

Venenum Bufonis, also called Chan Su, is a valuable traditional Chinese medicine which has been used for thousands years in China. Presently, several Chinese medicine formulations containing Venenum Bufonis are used in clinic for cancer therapy. Its main ingredients with antitumor activivities are a class of bufadienolides with an unsaturated six-membered lactone ring, represented by the following formulae:

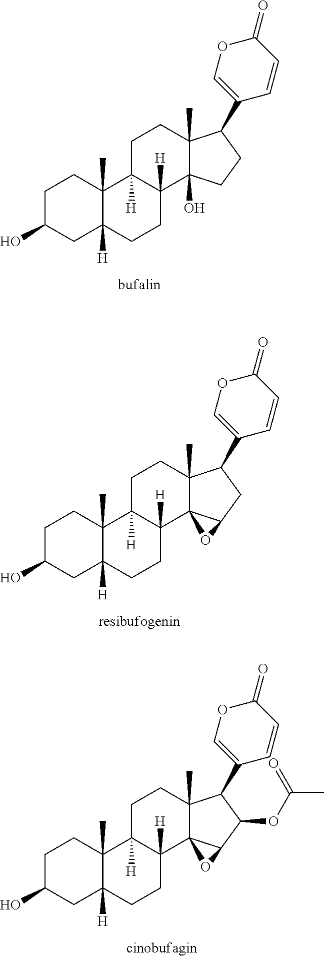

Therefore, it is important to synthesize new bufadienolide derivatives to clarify the antitumor structure-activity relationship and to find bufadienolide derivatives with stronger activity and lower toxicity. WO 2007081835 A2 discloses a class of cardiolide and bufadienolide compounds and their use for modulating the effects of local and systemic hypoxic events, and specifically relates to the following compounds:

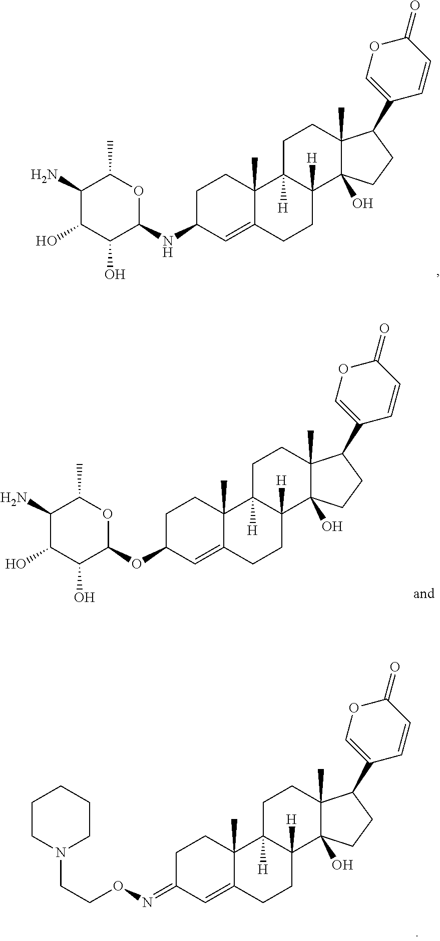

WO 2011085641 A1 discloses a class of bufalin derivatives and its use in the treatment of cancer, and specifically discloses the following compounds:

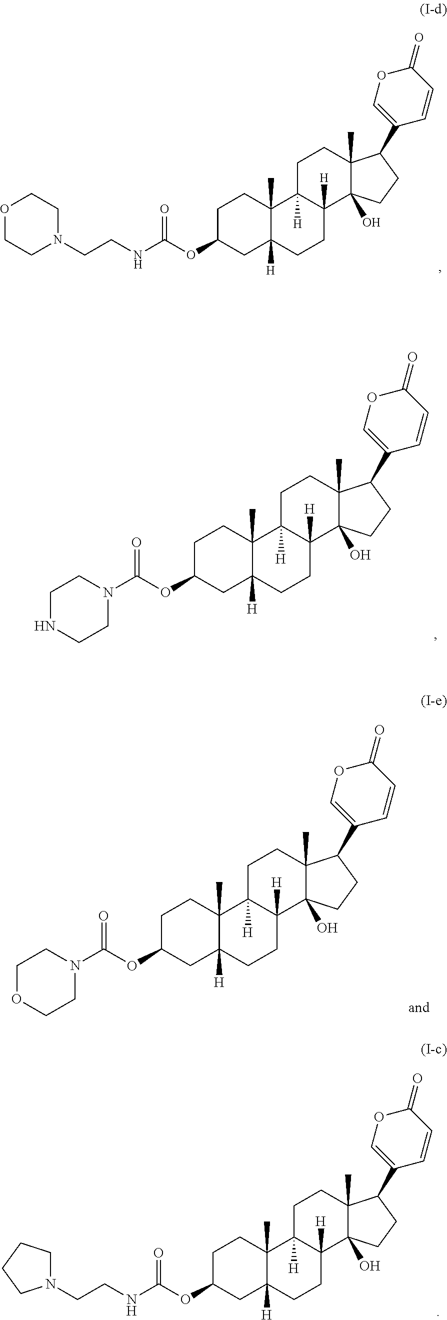

However, the phamaceutic activities of above compounds are still not satisfied. Thus, it is necessary to further develop bufalin derivatives to meet the requirement for a new drug.

### SUMMARY OF THE INVENTION

One object of the present invention is to provide a class of bufadienolide derivatives represented by formula I or pharmaceutically acceptable salts thereof. The bufadienolide derivatives have inhibitory activities against human-derived tumor cell lines, and thus might be used as a drug for treating malignancies.

Another object of the present invention is to provide a process for preparing the above bufadienolide derivatives.

Still another object of the present invention is to provide a pharmaceutic composition comprising a therapeutically effective amount of one or more selected from the group consisting of the bufadienolide derivatives and pharmaceutically acceptable salts thereof according to the present invention as the active components. Optionally, the pharmaceutic composition may further comprise pharmaceutically acceptable carriers, adjuvants or auxiliaries.

Still another object of the present invention is to provide the use of the above bufadienolide derivatives or the pharmaceutically acceptable salts thereof, and the pharmaceutic composition comparing the bufadienolide derivatives in preparing a medicament for treating malignancies.

Still another object of the present invention is to provide a pharmaceutic composition comprising a therapeutically effective amount of one or more selected from the group consisting of the bufadienolide derivatives and pharmaceutically acceptable salts thereof according to the present invention as the active components, and other pharmaceutically acceptable therapeutic agents, especially other antitumor drugs. Optionally, the pharmaceutic composition may further comprise pharmaceutically acceptable carriers, adjuvants or auxiliaries.

Still another object of the present invention is to provide a method for treating malignancies comprising administrating a subject having such need with a therapeutically effective amount of one or more selected from the group consisting the bufadienolide derivatives and pharmaceutically acceptable salts thereof according to the present invention, or the pharmaceutic composition comprising a therapeutically effective amount of one or more selected from the group consisting of the bufadienolide derivatives and pharmaceutically acceptable salts thereof according to the present invention as the active components according to the present invention.

In one aspect of the present invention, provided is a class of bufadienolide derivatives of the following formula I or pharmaceutically acceptable salts thereof,

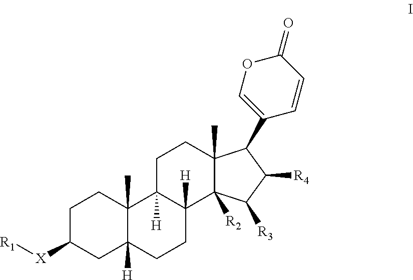

wherein,

X is O or NH;

R₁ is a group selected from any one of the following groups:

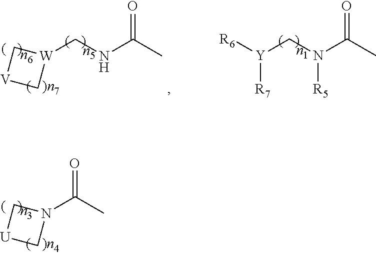

wherein,

n₅ is 0, 1, 2 or 3;

n₆ is 0, 1, 2, 3 or 4;

n₇ is 0, 1, 2, 3 or 4; and n₆ and n₇ are not 0 simultaneously;

W is CH;

V is R₁₅—N;

R₁₅ is H, C₁-C₆ alkyl, —C(═O)R₁₁, —SO₂—R₁₂ or an amino acid residue, preferably H, C₁-C₄ alkyl, —C(═O)R₁₁, or —SO₂—R₁₂, and most preferably H, methyl, ethyl;

n₁ is 1, 2 or 3;

Y is N;

R₅ is H or C₁-C₆ alkyl, preferably H or C₁-C₄ alkyl, and most preferably H, methyl or ethyl;

R₆ and R₇ are each independently H or C₁-C₆ alkyl, preferably H or C₁-C₄ alkyl, and most preferably H, methyl or ethyl;

n₃ is 0, 1, 2 or 3;

n₄ is 0, 1, 2, or 3; and n₃ and n₄ are not 0 simultaneously;

U is O, R₁₃—N or R₁₄—CH;

R₁₃ is H, C₁-C₆ alkyl, —C(═O)R₁₁, —SO₂—R₁₂ or an amino acid residue, preferably H, or C₁-C₄ alkyl, and most preferably H, methyl or ethyl;

R₁₁ and R₁₂ are each independently H, C₁-C₆ alkyl, C₃-C₇ cycloalkyl, C₁-C₆ alkyloxycarbonyl, benzyl, aryl, NH₂, amino substituted with C₁-C₆ alkyloxycarbonyl C₁-C₄ alkyl or C₃-C₇ cycloalkyl, amino substituted with benzyl or phenyl, C₁-C₆ alkyloxy or 5-7 membered heteroaryl, preferably H, C₁-C₄ alkyl, C₁-C₄ alkyloxycarbonyl, benzyl, aryl, NH₂, amino substituted with C₁-C₄ alkyloxycarbonyl C₁-C₂ alkyl or C₅-C₇ cycloalkyl, amino substituted with benzyl or phenyl, C₁-C₄ alkyloxy or pyridyl, and most preferably H, methyl, ethyl, methoxycarbonyl, ethoxycarbonyl, benzyl, phenyl, pyridyl, phenyl substituted with methyl, NO₂ or methoxycarbonyl, NH₂, amino substituted with ethoxycarboxylethyl, cyclohexyl or benzyl, methoxy, ethoxy or pyridyl;

R₁₄ is H, C₁-C₆ alkyl, hydroxyl, C₃-C₇ cycloalkyl, benzyl, aryl, NH₂, amino substituted with C₁-C₄ alkyl or hydroxyl C₁-C₄ alkyl, C₁-C₆ alkyloxy or 5-7 membered heteroaryl, preferably H, C₁-C₄ alkyl, hydroxyl, C₃-C₇ cycloalkyl, NH₂, amino substituted with C₁-C₄ alkyl or hydroxyl C₁-C₄ alkyl, or C₁-C₄ alkyloxy, and more preferably H, methyl, hydroxyl, hydroxylmethylamino, hydroxylethylamino or dimethylamino;

the aryl may be phenyl, naphthyl or biphenyl, or phenyl substituted with 1 to 4 substituents selected from the group consisting of halo, C₁-C₆ alkyl, CN, NO₂, NH₂, hydroxyl, hydroxyl C₁-C₄ alkyl, C₁-C₄ haloalkyl, carboxy, C₁-C₄ alkyloxy, C₁-C₄ haloalkyloxy, mercapto and C₁-C₄ alkyloxycarbonyl, preferably phenyl, or phenyl substituted with one substituent selected from the group consisting of C₁-C₄ alkyl, NO₂, NH₂, hydroxyl, hydroxyl C₁-C₄ alkyl, C₁-C₄ haloalkyl, carboxy, C₁-C₄ alkyloxy and C₁-C₄ alkyloxycarbonyl;

R₂ is —OH;

R₃ is —H;

R₄ is —H;

with the provision that the above compounds exclude the following compound:

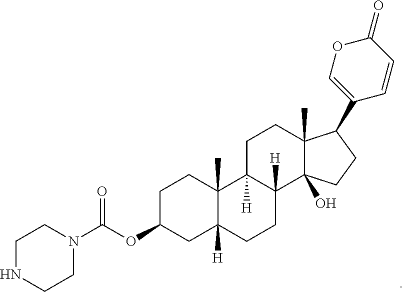

In the present invention, the term “aryl” refers to an aromatic ring group, preferably an aryl having 6-14 carbon atoms, and more preferably an aryl having 6-12 carbon atoms, such as phenyl, naphthyl, biphenyl, phenyl substituted with 1 to 4 substituents selected from the group consisting of halo, C₁-C₆ alkyl, CN, NO₂, NH₂, hydroxyl, hydroxyl C₁-C₄ alkyl, C₁-C₄ haloalkyl, carboxy, C₁-C₄ alkyloxy, C₁-C₄ haloalkyloxy, mercapto and C₁-C₄ alkyloxycarbonyl, such as 4-methylphenyl, 4-hydroxyphenyl, 2,3-dihydroxylphenyl, 3-hydroxylphenyl, 4-methoxyphenyl, 3-methoxy-4-hydroxylphenyl, 3,4-dimethoxyphenyl, more preferably phenyl, 4-hydroxyphenyl, 4-methoxyphenyl, 3-methoxy-4-hydroxylphenyl.

In the present invention, the term “C₁-C₆ alkyl” refers to a linear or branched alkyl having 1 to 6 carbon atoms in the backbone, and includes, but is not limited to, methyl, ethyl, propyl, isopropyl, n-butyl, isobutyl, sec-butyl, tert-butyl, pentyl, hexyl, etc., preferably isopropyl, n-butyl, isobutyl, sec-butyl, tert-butyl.

In the present invention, the term “C₁-C₆ alkyloxy” refers to a linear or branched alkyloxy having 1 to 6 carbon atoms in the backbone, and includes, but is not limited to, methoxy, ethoxy, propoxy, isopropoxy, butoxy, etc., preferably methoxy, ethoxy.

In the present invention, the term “C₃-C₇ cycloalkyl” refers to a cyclic alkyl having 3 to 7 carbon atoms on the ring, and includes, but is not limited to, cyclopropyl, cyclobutyl, cyclopentyl, cyclohexyl and cycloheptyl, preferably cyclopentyl, cyclohexyl and cycloheptyl.

In the present invention, the term “5-7 membered heteroaryl” refers to a 5-7 membered aromatic cyclic group having at least one heteroatom on the ring selected from N, O and S, and includes, but is not limited to, furyl, pyrryl, thienyl, oxazolyl, imidazolyl, pyrazolyl and pyridyl, preferably pyrryl, imidazolyl and pyridyl.

In the present invention, the amino acid residue refers to the amino acid part formed by bonding an amino acid onto the structure of a compound of the present invention through a condensation reaction, and the amino acid are commonly known in the art, and includes, but is not limited to, glycine, threonine, proline, tyrosine, tryptophane, aspartic acid, glutamic acid, etc., preferably glycine, proline, tyrosine, tryptophane.

In the present invention, the term “pharmaceutically acceptable salt” refers to a salt formed with an inorganic acid such as phosphoric acid, sulfuric acid, hydrochloric acid and the like, or an organic acid such as acetic acid, tartaric acid, citric acid, malic acid and the like, or an acidic amino acid such as aspartic acid, glutamic acid and the like, or a salt formed by reacting an inorganic alkali with an ester or amide formed with the above acid, such as sodium salt, potassium salt, calcium salt, aluminum salt and ammonium salt.

In a preferable embodiment of the present invention, provided is a class of bufadienolide derivatives of the following formula II or pharmaceutically acceptable salts thereof,

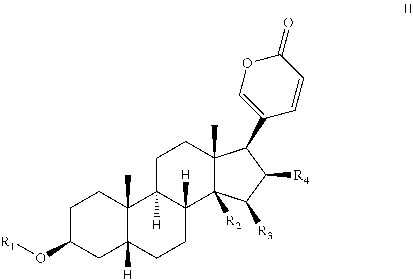

wherein, R₁, R₂, R₃ and R₄ are defined as those in formula I,

with the provision that the above compounds exclude the following compound:

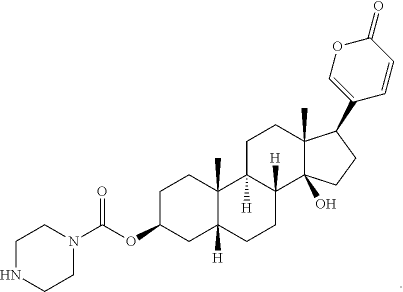

In a more preferable embodiment of the present invention, in the above formula II,

R₁ is

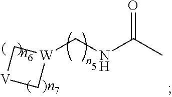

n₅ is 0, 1 or 2;

n₆ is 0, 1, 2, 3 or 4;

n₇ is 0, 1, 2, 3 or 4; and n₆ and n₇ are not 0 simultaneously;

W is CH;

V is R₁₅—N;

R₁₅ is H or C₁-C₆ alkyl.

In another preferable embodiment of the present invention, provided is a class of bufadienolide derivatives of the following formula III or pharmaceutically acceptable salts thereof,

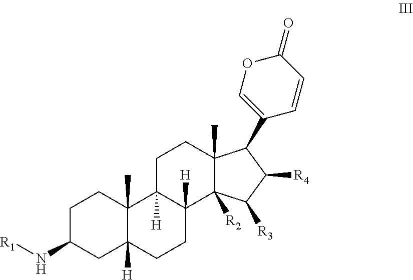

wherein, R₁, R₂, R₃ and R₄ are defined as those in formula I.

In the present invention, the specifically preferable compounds are:

| | | | | | I |
|---|---|---|---|---|---|
| 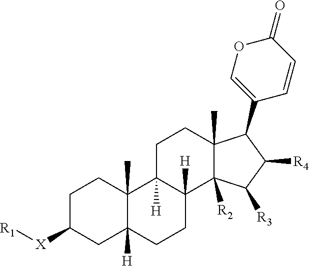 | | | | | |
| No. | X | R₁ | R₂ | R₃ | R₄ |
| II1B-01 | O | 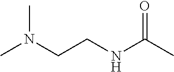 | OH | H | H |
| II1B-02 | O | 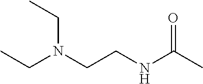 | OH | H | H |
| II1B-03 | O | 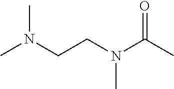 | OH | H | H |
| II1B-04 | O | 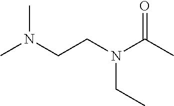 | OH | H | H |
| II1B-05 | O | 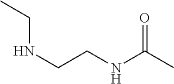 | OH | H | H |
| II1B-06 | O | 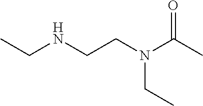 | OH | H | H |
| II1B-07 | O | 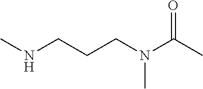 | OH | H | H |
| II1B-08 | O | 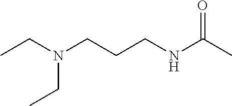 | OH | H | H |
| II1B-09 | O | 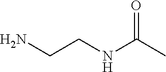 | OH | H | H |
| II1B-10 | O | 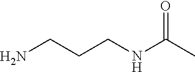 | OH | H | H |
| II1D-01 | O | 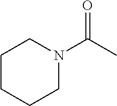 | OH | H | H |
| II1D-03 | O | 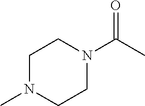 | OH | H | H |
| II1D-04 | O | 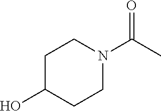 | OH | H | H |
| II1D-06 | O | 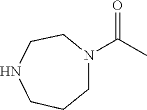 | OH | H | H |
| II1D-07 | O | 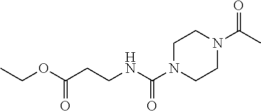 | OH | H | H |
| II1D-08 | O | 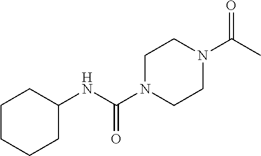 | OH | H | H |
| II1D-09 | O | 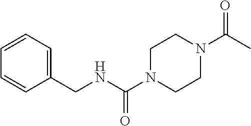 | OH | H | H |
| II1D-10 | O | 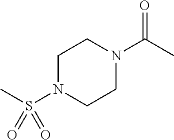 | OH | H | H |
| II1D-11 | O | 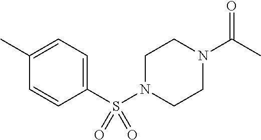 | OH | H | H |
| II1D-12 | O | 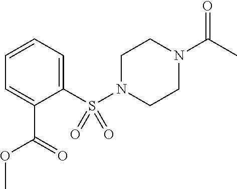 | OH | H | H |
| II1D-13 | O | 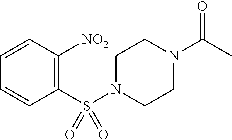 | OH | H | H |
| II1D-14 | O | 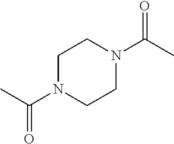 | OH | H | H |
| II1D-15 | O | 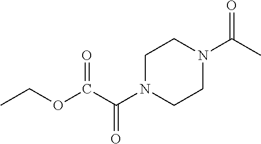 | OH | H | H |
| II1D-16 | O | 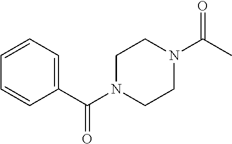 | OH | H | H |
| II1D-17 | O | 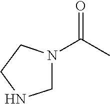 | OH | H | H |
| II1D-20 | O | 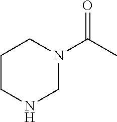 | OH | H | H |
| II1D-22 | O | 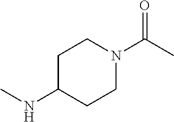 | OH | H | H |
| II1D-23 | O | 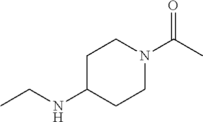 | OH | H | H |
| II1D-24 | O | 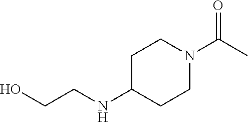 | OH | H | H |
| II1D-25 | O | 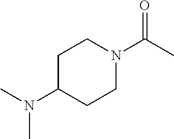 | OH | H | H |
| II1E-01 | O | 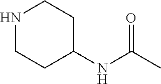 | OH | H | H |
| II1E-02 | O | 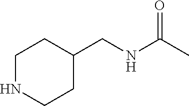 | OH | H | H |
| II1E-03 | O | 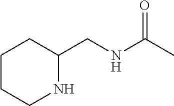 | OH | H | H |
| II1E-04 | O | 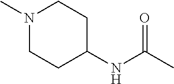 | OH | H | H |
| II1E-05 | O | 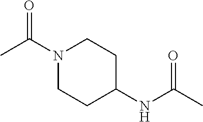 | OH | H | H |
| III1D-02 | NH | 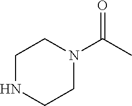 | OH | H | H |
| III1D-03 | NH | 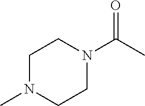 | OH | H | H |
| III1D-04 | NH | 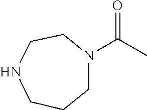 | OH | H | H |
| III1E-01 | NH | 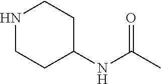 | OH | H | H |
| III1E-02 | NH | 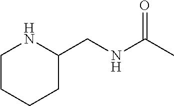 | OH | H | H |

In the second aspect of the present invention, provided is a process for preparing the bufadienolide derivatives according to the present invention, including the following methods:

Method A: in the case of when X is 0

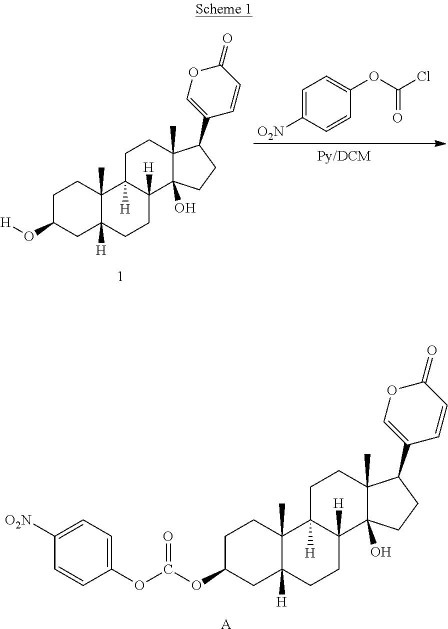

(1) as shown in Scheme 1, compound I is reacted with p-nitrophenyl chloroformate through an esterification to give an intermediate A, wherein the specific reaction conditions for the esterification are the common selections for a person skilled in the art, for example, the esterification may be performed in the presence of an alkali such as triethylamine, diisopropylethylamine, pyridine (Py) and 4-(N,N-dimethyl)aminopyridine (DMAP) in an organic solvent such as dichloromethane (DCM);

(2) the intermediate A is reacted with an amine corresponding to the final product

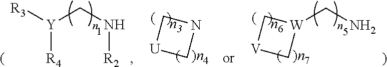

through a substitution to provide a carbamate compound of formula I, wherein the substitution may be performed under conditions which are common selections for a person skilled in the art, for example, at room temperature in the present of an alkali such as triethylamine, K₂CO₃ and pyridine;

(3) the carbamate compound synthesized in step (2) is reacted with an acyl chloride, an isocyanate or a sulfonyl chloride (R₁₂SO₃Cl, wherein R₁₂ is defined as that of formula I) corresponding to the final product to provide an acylate of the carbamate compound, wherein the acylation may be performed under conditions which are common selections for a person skilled in the art, for example, at room temperature in a solvent such as dichloromethane in the present of an alkali such as triethylamine, K₂CO₃ and pyridine;

Method B: in the case of when X is N, the process is performed as shown in the following Scheme 2:

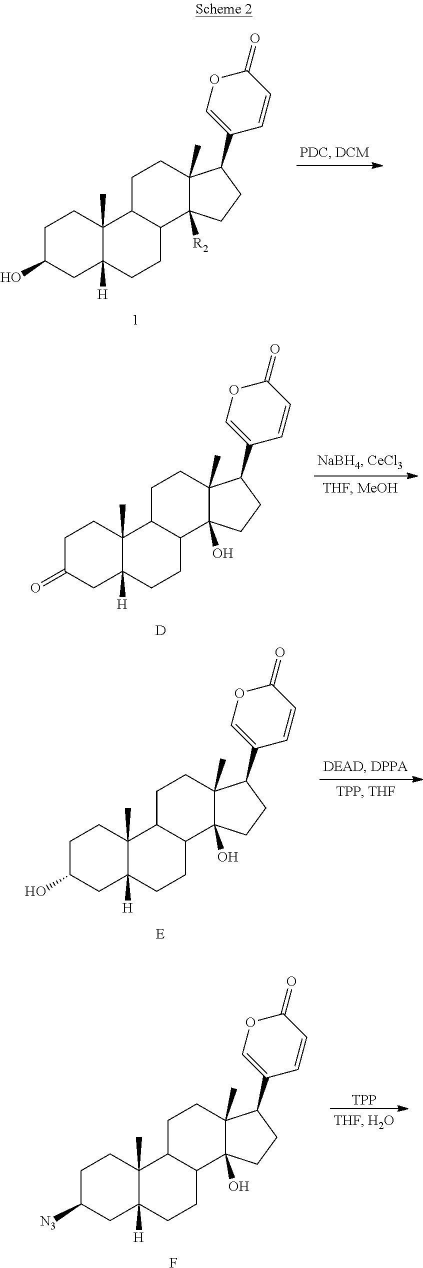

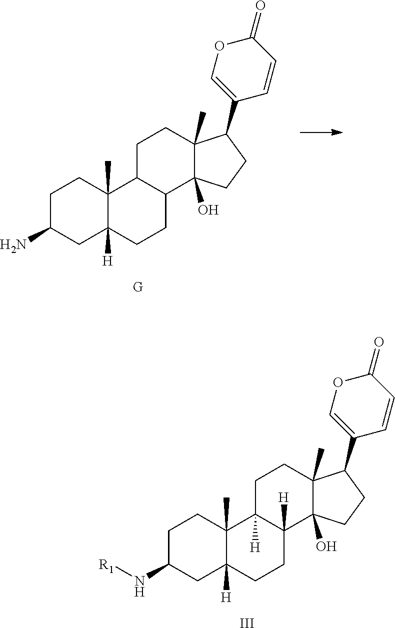

(1) compound I is converted through an oxidation into an intermediate D, wherein the oxidation may be performed under specific conditions which are common selections for a person skilled in the art, for example, in an organic solvent such as dichloromethane(DCM) using an oxidant such as pyridinium dichromate (PDC);

(2) the compound D is converted through a reduction into an intermediate E, wherein the reduction may be performed under specific conditions which are common selections for a person skilled in the art, for example, in an organic solvent such as a mixture of methanol (MeOH)-tetrahydrofuran (THF) in the presence of a catalyst such as cerous chloride (CeCl₃) using a reductant such as NaBH₄;

(3) The compound E is converted through an azidation into an intermediate F, wherein the azidation may be performed under specific conditions which are common selections for a person skilled in the art, for example, in an organic solvent such as tetrahydrofuran using an azide reagent such as diethyl azodicarboxylate-diphenylphosphoryl azide-triphenylphosphine;

(4) the compound F is converted through a reduction into an intermediate G, wherein the reduction may be performed under specific conditions which are common selections for a person skilled in the art, for example, in an organic solvent such as a mixture of tetrahydrofuran-water using a reductant such as triphenylphosphine;

wherein, each of the substitutents is defined as that of formula I.

The bufadienolide derivatives or pharmaceutically acceptable salts thereof obtained according to the present invention may be administered to a human via oral, rectal, parenteral (intravenous, intramuscular or subcutaneous), local administration (powder, ointment or drop) routes.

The solid dosage forms for oral administration include capsules, tablets, pills, powders and granules. In these solid dosage forms, the active compound is mixed with at least one commonly used inert excipient (or vehicle) such as sodium citrate or dicalcium phosphate, or with the following components: (a) fillers or compatibilizers, such as starch, lactose, sucrose, glucose, mannitol and silicic acid; (b) binders, such as hydroxymethylcellulose, alginates, gelatin, polyvinylpyrrolidone, sucrose and arabic gum; (c) humectants, such as glycerol; (d) disintegrants, such as agar, calcium carbonate, amylum solani or tapioca, alginic acid, some composite silicates and sodium carbonate; (e) suspended dissolving agents such as paraffin; (f) adsorption enhancers, such as quaternary ammonium compounds; (g) wetting agents, such as cetyl alcohol and glyceryl monostearate; (h) adsorbents, such as kaolin; and (i) lubricants, such as talc, calcium stearate, magnesium stearate, solid polyethylene glycol, sodium laurylsulfate, or mixtures thereof. In the capsules, tablets and pills, the preparation can also comprise a buffer.

The solid dosage forms such as tablets, rotulas, capsules, pills and granules may be prepared using a coating or a shell material, such as casing or other materials commonly known in the art. They may comprise an opague agent, and the active compounds or compounds may be released in a certain portion of the digestive tract in a delayed manner. The examples of the usable embedding component are polymeric substances and waxes. If necessary, the active compound may also be formed with one or more of the above excipients into a microcapsule form.

Liquid dosage forms for oral administration include pharmaceutically acceptable emulsions, solutions, suspensions, syrups or tinctures. Besides the active compounds, the liquid preparation may comprise an inert diluent commonly used in the art such as water or other solvents, a solubilizer and an emulsifier, such as ethanol, isopropanol, ethyl carbonate, ethyl acetate, propylene glycol, 1,3-butylene glycol, dimethylformamide, and oils, especially cotton seed oil, peanut oil, maize germ oil, olive oil, castor oil and sesame oil, or mixtures thereof.

Besides these inert diluents, the above composition may also comprise an adjuvant, such as a wetting agent, an emulsifier and a suspending agent, a sweetening agent, a correctant and a perfume.

Besides the active compounds, the suspension may comprise a suspending agent, such as ethoxylated isooctadecanol, polyoxyethylene sorbitol and sorbitan esters, microcrystalline cellulose, aluminium methoxide and agar, or mixtures thereof.

Compositions for parenteral injection may include physiologically acceptable sterile aqueous or nonaqueous solution, dispersion, suspension or emulsion, and sterile powders to be redissolved into sterile and injectable solution or dispersion. Suitable aqueous and nonaqueous vehicles, diluents, solvents or excipients include water, ethanol, polyols, and suitable mixtures thereof.

The dosage forms of the compound according to the present invention for local administration include ointments, powders, sprays and inhalers. The active component(s) is/are mixed under a sterile condition with physiologically acceptable vehicles, preservatives, buffers, or if necessary, possibly required propellants.

Therefore, in the third aspect of the present invention, provided is a pharmaceutic composition comprising a therapeutically effective amount of one or more selected from the group consisting of the bufadienolide derivatives and pharmaceutically acceptable salts thereof according to the present invention as the active components, and optionally, pharmaceutically acceptable vehicles, excipients, adjuvants, auxiliaries and/or diluents.

In the fourth aspect of the present invention, provided is the use of the bufotalins or pharmaceutically acceptable salts thereof and the pharmaceutic composition comprising the same according to the present invention in preparation of a medicament for treating malignancies, which comprises administering a subject having such need with a therapeutically effective amount of one or more selected from the group consisting the bufadienolide derivatives and pharmaceutically acceptable salts thereof according to the present invention, or the pharmaceutic composition comprising a therapeutically effective amount of one or more selected from the group consisting of the bufadienolide derivatives and pharmaceutically acceptable salts thereof according to the present invention as the active components according to the present invention. The compounds or pharmaceutically acceptable salts thereof according to the present invention may be administered alone, or in combination with other pharmaceutically acceptable therapeutical agents, especially other antitumor agents. The therapeutical agent includes, but is not limited to, antitumor agents acting on the chemical structure of DNA, such as Cisplatin, antitumor agents affecting the synthesis of nucleic acid, such as methotrexate (MTX), 5-fluorouracil (5FU), etc., antitumor agents affecting the transcription of nucleic acid, such as adriamycin, pharmorubicin, aclacinomycin, mithramycin, etc., antitumor agents acting on the systhesis of tubulin, such as paclitaxel, vinorelbine, etc., aromatase inhibitors such as aminoglutethimide, lentaron, letrozole, arimidex, etc., inhibitors to cell signaling pathway such as epidermal growth factor receptor inhibitor Imatinib, Gefitinib, Erlotinib, Lapatinib, etc. The various components to be combined may be administered simultaneously or in sequence, in a single preparation or in separate preparations. The combination includes not only the combination of the compound according to the present invention and one of other active agents, but also the combination of the compound according to the present invention and two or more of other active agents.

Therefore, in the fifth aspect of the present invention, provided is a pharmaceutic composition comprising a therapeutically effective amount of one or more selected from the group consisting of the bufadienolide derivatives and pharmaceutically acceptable salts thereof according to the present invention as the active components, and other pharmaceutically acceptable therapeutic agents, especially other antitumor drugs. Optionally, the pharmaceutic composition may further comprise pharmaceutically acceptable vehicles, excipients, adjuvants, auxiliaries and/or diluents.

In the sixth aspect of the present invention, provided is a method for treating malignancies comprising administrating a subject having such need with a therapeutically effective amount of one or more selected from the group consisting of the bufotalins and pharmaceutically acceptable salts thereof according to the present invention, or the pharmaceutic composition according to the present invention comprising a therapeutically effective amount of one or more selected from the group consisting of the bufadienolide derivatives and pharmaceutically acceptable salts thereof according to the present invention as the active components.

In the present invention, the malignancies include, but are not limited to, liver cancer, lung cancer, breast cancer, stomach cancer, esophageal cancer, colon cancer, leukemia, lymph cancer, prostate cancer, renal cancer, skin cancer, pancreatic cancer, ovarian cancer, brain cancer, bone cancer, fibrosarcoma and the like, preferably liver cancer, lung cancer, colon cancer, prostate cancer, stomach cancer, leukemia and the like.

The present invention designs and synthesizes a class of novel bufadienolide derivatives which have inhibitory activities against tumor cell lines and thus can be used as a drug for treating malignancies. The compounds of the present invention have a simple synthesizing route, are easy to be prepared, and abound with the synthesizing raw materials.

### BEST MODE FOR CARRYING OUT THE INVENTION

Hereinafter, the present invention will be further illustrated with reference to specific examples, but the present invention is not limited thereto. The experimental operation of the present invention is a general procedure, and not limited to the compounds mentioned in the invention.

In the following preparation examples, ¹H-NMR was measured on a Varian Mercury AMX300. MS was measured on a VG ZAB-HS, or a VG-7070, and a Esquire 3000 plus-01005. All the solvents were subjected to redistillation before use, and all the anhydrous solvent are obtained by a drying process according to the standard method. Unless otherwise indicated, all the reactions were performed under argon and traced with TLC, and the posttreatment included washing with saturated sodium chloride aqueous solution and drying over anhydrous magnesium sulfate. Unless otherwise indicated, each purification of the product adopted a column chromatography using a silica gel of 200-300 mesh, the silica gel used include GF₂₅₄ of 200-300 mesh produced by Qingdao Haiyang Chemical Plant or Yantai Yuanbo Silica Gel Company. Venenum Bufonis was extracted with 95% ethanol, concentrated, and passed twice through column chromatography to give a crude bufalin. The crude was subjected to a recrystallization from ethanol to obtain bufalin.

### Example 1

### Preparation of Compound II1B-01

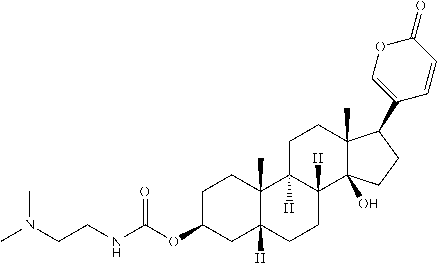

In a 50 mL round bottom flask, p-nitrophenyl chloroformate (1.206 g, 6 mmol) was dissolved in 10 mL of anhydrous dichloromethane, followed by addition of dry pyridine (0.67 mL). A white precipitate appeared immediately. A solution of bufalin (2 mmol) in dichloromethane (10 mL) was dropwisely added under nitrogen, and stirred at room temperature for 6 hours. After completion of the reaction, the reaction mixture was washed twice with water, dried over anhydrous sodium sulfate, and concentrated under reduced pressure. The residue passed through silica gel column chromatography (90:10, petroleum ether/acetone) to give an intermediate A.

In a 10 mL round bottom flask, the intermediate A was dissolved in 3 mL of dichloromethane, followed by addition of triethylamine (35 μL). N,N-dimethylethylenediamine (6 mmol) was added, and stirred at room temperature for 2 hours (hereinafter, the reaction time of similar reactions is determined by thin-layer chromatography). After completion of the reaction, the reaction mixture was washed once with saturated sodium carbonate aqueous solution, and then repeatedly with water until the solution is clear, dried over anhydrous sodium sulfate, and concentrated under reduced pressure. The residue passed through silica gel column chromatography (petroleum ether/acetone/ammonia water, 50:50:0.5) to give the product II1B-01 with a yield of 91%. ¹H NMR (CDCl₃, 300 MHz) δ: 7.83 (dd, 1H, J=9.6, 2.4 Hz), 7.23 (d, 1H, J=2.4 Hz), 6.25 (d, 1H, J=9.6 Hz), 5.00 (br s, 1H), 3.40 (s, 2H), 2.94 (s, 3H), 2.77 (t, 2H, J=6.3 Hz), 2.46 (s, 3H), 1.08-2.21 (m, 22H), 0.95 (s, 3H), 0.70 (s, 3H); ¹³C NMR (CDCl₃, 75 MHz) δ: 16.7, 21.5, 21.6, 24.1, 25.5, 26.7, 28.9, 29.8, 30.9, 32.9, 35.4, 36.0, 36.4, 37.4, 37.4, 41.0, 42.5, 48.5, 48.6, 49.8, 51.4, 71.4, 85.4, 115.4, 122.9, 147.0, 148.7, 155.5, 162.6; ESI-MS (m/z) 501.4 [M+1]⁺.

### Example 2

### Preparation of Compound II1B-02

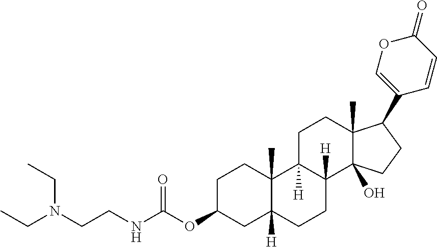

The reaction operations were as the preparation of compound II1B-01, except that N,N-diethylethylenediamine was used instead of N,N-dimethylethylenediamine. The eluent for the silica gel column chromatography is petroleum ether/acetone/ammonia water (50:50:0.5), and the yield is 78%. ¹H NMR (CDCl₃, 300 MHz) δ: 7.84 (dd, 1H, J=9.6, 2.4 Hz), 7.22 (d, 1H, J=2.4 Hz), 6.25 (d, 1H, J=9.6 Hz), 5.21 (br s, 1H), 3.23 (d, 2H, J=5.4 Hz), 2.55 (m, 6H), 1.02 (t, 6H, J=7.2 Hz), 1.09-2.26 (m, 22H), 0.94 (s, 3H), 0.69 (s, 3H); ¹³C NMR (CDCl₃, 75 MHz) δ: 11.8, 11.8, 16.7, 21.5, 21.6, 23.9, 25.5, 26.6, 28.9, 29.9, 30.9, 32.9, 35.3, 36.0, 36.9, 38.7, 41.0, 42.5, 47.1, 47.1, 48.5, 51.4, 52.1, 70.7, 85.5, 115.4, 122.9, 147.1, 148.7, 156.6, 162.6; ESI-MS (m/z) 529.5 [M+1]⁺.

### Example 3

### Preparation of Compound II1B-03

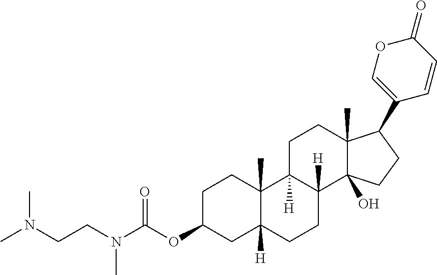

The reaction operations were as the preparation of compound II1B-01, except that N,N,N′-trimethylethylenediamine was used instead of N,N-dimethylethylenediamine. The eluent for the silica gel column chromatography is petroleum ether/acetone/triethylamine (60:40:0.5), and the yield is 90%. ¹H NMR (CDCl₃, 300 MHz) δ: 7.83 (dd, 1H, J=9.6, 2.4 Hz), 7.22 (d, 1H, J=2.4 Hz), 6.24 (d, 1H, J=9.6 Hz), 4.99 (br s, 1H), 3.36 (t, 2H, J=7.2 Hz), 3.36 (s, 3H), 2.44 (t, 2H, J=7.2 Hz), 2.26 (s, 6H), 1.20-2.20 (m, 22H), 0.94 (s, 3H), 0.69 (s, 3H); ¹³C NMR (CDCl₃, 75 MHz) δ: 16.7, 21.5, 21.6, 24.1, 25.5, 26.6, 28.9, 29.8, 30.9, 32.9, 34.6, 35.3, 36.0, 37.4, 41.0, 42.5, 47.1, 45.9, 48.5, 51.4, 57.0, 57.4, 71.2, 85.4, 115.4, 122.9, 147.1, 148.7, 156.1, 162.6; ESI-MS (m/z) 515.3 [M+1]⁺.

### Example 4

### Preparation of Compound II1B-04

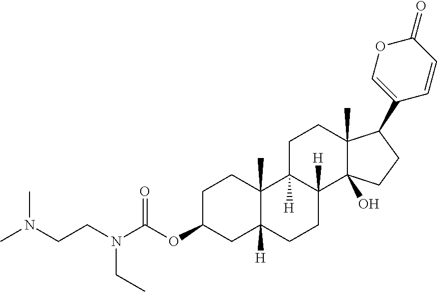

The reaction operations were as the preparation of compound II1B-01, except that N,N-dimethyl-N′-ethylethylenediamine was used instead of N,N-dimethylethylenediamine. The eluent for the silica gel column chromatography is petroleum ether/acetone/triethylamine (60:40:0.5), and the yield is 73%. ¹H NMR (CDCl₃, 300 MHz) δ: 7.83 (dd, 1H, J=9.6, 2.4 Hz), 7.23 (d, 1H, J=2.4 Hz), 6.25 (d, 1H, J=9.6 Hz), 5.01 (br s, 1H), 3.31 (m, 4H), 2.46 (t, 2H, J=6.9 Hz), 2.27 (s, 6H), 1.21-2.20 (m, 22H), 1.13 (t, 3H, J=7.2 Hz), 0.94 (s, 3H), 0.69 (s, 3H); ESI-MS (m/z) 529.4 [M+1]⁺.

### Example 5

### Preparation of Compound II1B-05

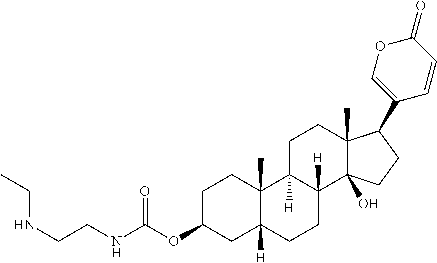

The reaction operations were as the preparation of compound II1B-01, except that N-ethylethylenediamine was used instead of N,N-dimethylethylenediamine. The eluent for the silica gel column chromatography is petroleum ether/acetone/ammonia water (50:50:0.5), and the yield is 72%. ¹H NMR (CDCl₃, 300 MHz) δ: 7.83 (dd, 1H, J=9.6, 2.4 Hz), 7.22 (d, 1H, J=2.4 Hz), 6.25 (d, 1H, J=9.6 Hz), 4.98 (br s, 1H), 3.27 (q, 2H, J=6.0 Hz), 2.75 (t, 2H, J=6.0 Hz), 2.65 (t, 2H, J=6.0 Hz), 1.12-2.45 (m, 22H), 1.10 (t, 3H, J=7.2 Hz), 0.93 (s, 3H), 0.69 (s, 3H); ESI-MS (m/z) 501.4 [M+1]⁺.

### Example 6

### Preparation of Compound II1B-06

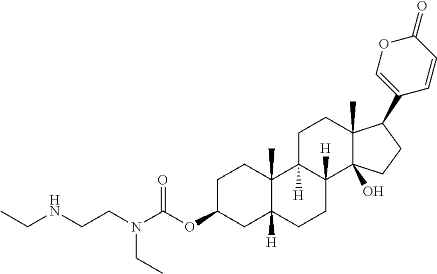

The reaction operations were as the preparation of compound II1B-01, except that N,N′-diethylethylenediamine was used instead of N,N-dimethylethylenediamine. The eluent for the silica gel column chromatography is petroleum ether/acetone/ammonia water (50:50:0.5), and the yield is 91%. ¹H NMR (CDCl₃, 300 MHz) δ: 7.83 (dd, 1H, J=9.6, 2.4 Hz), 7.23 (d, 1H, J=2.4 Hz), 6.25 (d, 1H, J=9.6 Hz), 5.01 (br s, 1H), 3.34 (m, 4H), 2.67 (t, 2H, J=7.2 Hz), 1.16-2.24 (m, 22H), 1.11 (t, 6H, J=7.2 Hz), 0.95 (s, 3H), 0.70 (s, 3H); ¹³C NMR (CDCl₃, 75 MHz) δ: 14.0, 15.4, 16.7, 21.5, 21.6, 24.1, 25.6, 26.7, 28.9, 29.8, 31.0, 32.9, 35.4, 36.0, 37.5, 41.0, 42.5, 42.7, 44.2, 47.0, 48.1, 48.5, 51.4, 71.1, 85.4, 115.4, 122.9, 147.1, 148.7, 156.1, 162.6; ESI-MS (m/z) 529.5 [M+1]⁺.

### Example 7

### Preparation of Compound II1B-09

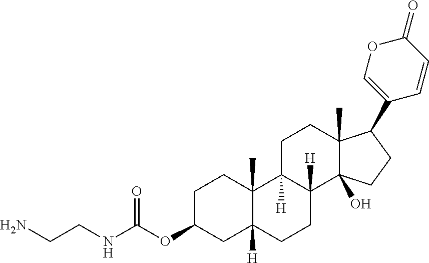

The reaction operations were as the preparation of compound II1B-01, except that 1,2-ethylenediamine was used instead of N,N-dimethylethylenediamine. The eluent for the silica gel column chromatography is petroleum ether/acetone/ammonia water (50:50:0.5), and the yield is 85%. ¹H NMR (CDCl₃, 300 MHz) δ: 7.83 (d, 1H, J=9.7 Hz), 7.22 (s, 1H), 6.24 (d, 1H, J=9.7 Hz), 4.98 (s, 1H), 3.40 (t, 2H, J=6.6 Hz), 3.21 (t, 2H, J=6.6 Hz), 2.44 (m, 1H), 1.13-2.50 (m, 21H), 0.90 (s, 3H), 0.67 (s, 3H); ESI-MS (m/z) 473.3 [M+1]⁺.

### Example 8

### Preparation of Compound II1B-10

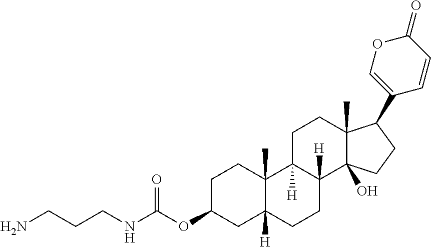

The reaction operations were as the preparation of compound II1B-01, except that 1.3-propylenediamine was used instead of N,N-dimethylethylenediamine. The eluent for the silica gel column chromatography is petroleum ether/acetone/ammonia water (50:50:0.5), and the yield is 85%. ¹H NMR (CDCl₃, 300 MHz) δ: 7.84 (d, 1H, J=9.7 Hz), 7.23 (s, 1H), 6.26 (d, 1H, J=9.7 Hz), 5.03 (s, 1H), 4.98 (s, 1H), 3.27 (m, 2H), 2.78 (t, 2H, J=6.6 Hz), 2.45 (m, 1H), 1.13-2.48 (m, 23H), 0.94 (s, 3H), 0.70 (s, 3H); ESI-MS (m/z) 487.3 [M+1]⁺.

### Example 9

### Preparation of Compound II1B-07

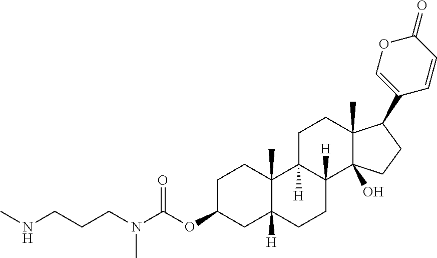

The reaction operations were as the preparation of compound II1B-01, except that N,N′-dimethylpropylenediamine was used instead of N,N-dimethylethylenediamine. The eluent for the silica gel column chromatography is petroleum ether/acetone/ammonia water (50:50:0.5), and the yield is 91%. ¹H NMR (CDCl₃, 300 MHz) δ: 7.83 (d, 1H, J=9.6 Hz), 7.22 (s, 1H), 6.25 (d, 1H, J=9.6 Hz), 4.99 (br s, 1H), 3.32 (t, 2H, J=6.0 Hz), 2.89 (s, 3H), 2.58 (t, 2H, J=6.3 Hz), 2.42 (s, 3H), 1.16-2.41 (m, 24H), 0.94 (s, 3H), 0.69 (s, 3H); ESI-MS (m/z) 515.3 [M+1]⁺.

### Example 10

### Preparation of Compound II1B-08

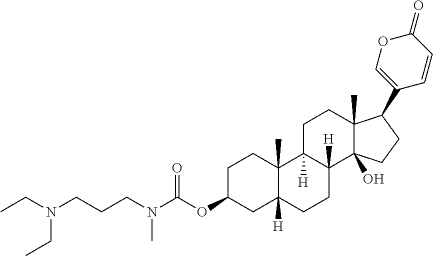

The reaction operations were as the preparation of compound II1B-01, except that N,N-diethyl-N′-methylpropylenediamine was used instead of N,N-dimethylethylenediamine. The eluent for the silica gel column chromatography is petroleum ether/acetone/ammonia water (50:50:0.5), and the yield is 86%. ¹H NMR (CDCl₃, 300 MHz) δ: 7.83 (dd, 1H, J=9.6, 2.4), 7.22 (d, 1H, J=2.4 Hz), 6.25 (d, 1H, J=9.6 Hz), 4.96 (br s, 1H), 3.23 (m, 2H), 2.52 (m, 6H), 1.16-2.24 (m, 24H), 1.05 (t, 6H, J=7.2 Hz), 0.93 (s, 3H), 0.69 (s, 3H); ESI-MS (m/z) 557.4 [M+1]⁺.

### Example 11

### Preparation of Compound II1D-01

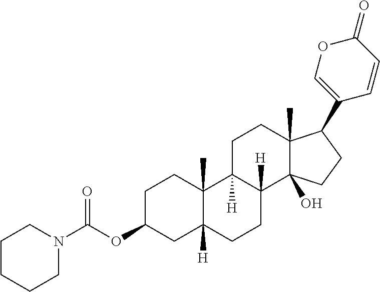

The reaction operations were as the preparation of compound II1B-01, except that piperidine was used instead of N,N-dimethylethylenediamine. The eluent for the silica gel column chromatography is petroleum ether/acetone (80:20), and the yield is 74%. ¹H NMR (CDCl₃, 300 MHz) δ: 7.83 (dd, 1H, J=9.6, 2.7 Hz), 7.23 (d, 1H, J=2.7 Hz), 6.25 (d, 1H, J=9.6 Hz), 5.01 (br s, 1H), 3.41 (m, 4H), 1.17-2.49 (m, 28H), 0.95 (s, 3H), 0.70 (s, 3H); ESI-MS (m/z) 520.4 [M+23]⁺.

### Example 12

### Preparation of Compound II1D-04

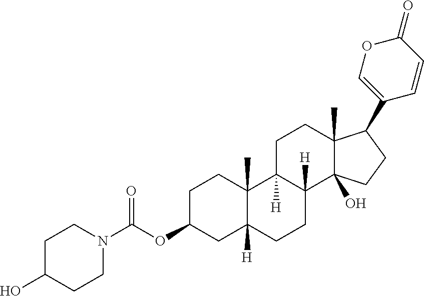

The reaction operations were as the preparation of compound II1B-01, except that 4-hydroxypiperidine was used instead of N,N-dimethylethylenediamine. The eluent for the silica gel column chromatography is petroleum ether/acetone (70:30), and the yield is 97%. ¹H NMR (CDCl₃, 300 MHz) δ: 7.83 (dd, 1H, J=9.6, 2.4 Hz), 7.23 (d, 1H, J=2.4 Hz), 6.25 (d, 1H, J=9.6 Hz), 5.00 (br s, 1H), 3.88 (m, 3H), 3.09 (m, 2H), 1.17-2.49 (m, 26H), 0.95 (s, 3H), 0.70 (s, 3H); ¹³C NMR (CDCl₃, 75 MHz) δ: 16.7, 21.5, 21.6, 24.1, 25.5, 26.7, 28.9, 29.9, 31.0, 33.0, 34.3, 34.3, 35.4, 36.0, 37.4, 41.0, 41.4, 41.4, 42.5, 48.6, 51.4, 67.7, 71.3, 85.5, 115.5, 122.9, 147.1, 148.8, 155.5, 162.7; ESI-MS (m/z) 514.4 [M+1]⁺.

### Example 13

### Preparation of Compound II1D-05

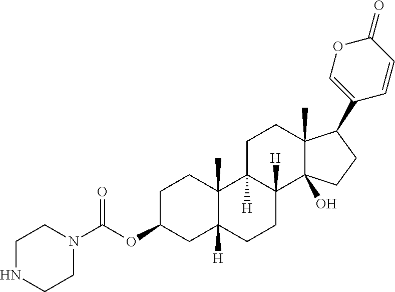

The reaction operations were as the preparation of compound II1B-01, except that piperazine was used instead of N,N-dimethylethylenediamine. The eluent for the silica gel column chromatography is petroleum ether/acetone/ammonia water (50:50:0.5), and the yield is 87%. ¹H NMR (CDCl₃, 300 MHz) δ: 7.83 (dd, 1H, J=9.6, 2.4 Hz), 7.23 (d, 1H, J=2.4 Hz), 6.25 (d, 1H, J=9.6 Hz), 5.02 (br s, 1H), 3.45 (t, 4H, J=5.1 Hz), 2.84 (t, 4H, J=5.1 Hz), 1.06-2.49 (m, 22H), 0.95 (s, 3H), 0.70 (s, 3H); ¹³C NMR (CDCl₃, 75 MHz) δ: 16.7, 21.5, 21.6, 24.1, 25.5, 26.6, 28.9, 29.8, 31.0, 32.9, 35.4, 36.0, 37.4, 41.0, 42.5, 44.8, 44.8, 45.9, 45.9, 48.5, 51.4, 71.3, 85.4, 115.4, 122.9, 147.1, 148.7, 155.2, 162.6; ESI-MS (m/z) 499.3 [M+1]⁺.

### Example 14

### Preparation of Compound II1D-03

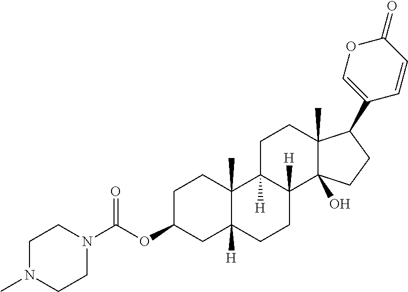

The reaction operations were as the preparation of compound II1B-01, except that N-methylpiperazine was used instead of N,N-dimethylethylenediamine. The eluent for the silica gel column chromatography is petroleum ether/acetone/ammonia water (80:20:0.5), and the yield is 83%. ¹H NMR (CDCl₃, 300 MHz) δ: 7.83 (dd, 1H, J=9.6, 2.7 Hz), 7.23 (d, 1H, J=2.7 Hz), 6.25 (d, 1H, J=9.6 Hz), 5.02 (br s, 1H), 3.49 (t, 4H, J=4.8 Hz), 2.39 (t, 4H, J=4.8 Hz), 2.30 (s, 3H), 1.10-2.48 (m, 22H), 0.95 (s, 3H), 0.70 (s, 3H); ESI-MS (m/z) 513.3 [M+1]⁺.

### Example 15

### Preparation of Compound II1E-01

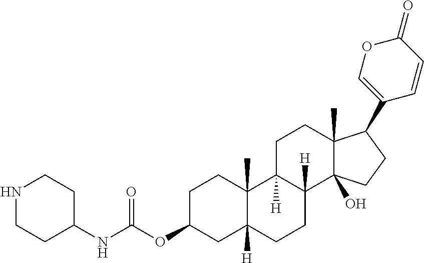

The reaction operations were as the preparation of compound II1B-01, except that 4-aminopiperidine was used instead of N,N-dimethylethylenediamine. The eluent for the silica gel column chromatography is petroleum ether/acetone/ammonia water (50:50:0.5), and the yield is 81%. ¹H NMR (CDCl₃, 400 MHz) δ: 7.85 (dd, 1H, J=9.6, 2.5 Hz), 7.22 (d, 1H, J=2.5 Hz), 6.25 (d, 1H, J=9.6 Hz), 5.60 (br s, 1H), 5.00 (br s, 1H), 4.09 (br s, 2H), 2.83 (m, 5H), 1.04-2.48 (m, 26H), 0.95 (s, 3H), 0.69 (s, 3H); ¹³C NMR (CDCl₃, 75 MHz) δ: 16.7, 21.6, 21.6, 24.1, 25.5, 26.7, 28.9, 29.9, 30.9, 33.0, 35.4, 35.6, 35.6, 36.1, 37.4, 41.1, 42.6, 42.9, 42.9, 48.6, 48.9, 51.4, 71.2, 85.5, 115.5, 122.9, 147.0, 148.7, 155.2, 162.6; ESI-MS (m/z) 513.3 [M+1]⁺.

### Example 16

### Preparation of Compound II1E-02

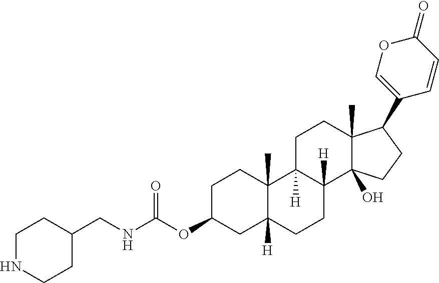

The reaction operations were as the preparation of compound II1B-01, except that 4-aminomethylpiperidine was used instead of N,N-dimethylethylenediamine. The eluent for the silica gel column chromatography is petroleum ether/acetone/ammonia water (50:50:0.5), and the yield is 86%. ¹H NMR (CDCl₃, 300 MHz) δ: 7.83 (dd, 1H, J=9.6, 2.4 Hz), 7.23 (d, 1H, J=2.4 Hz), 6.25 (d, 1H, J=9.6 Hz), 5.00 (br s, 1H), 4.14 (br s, 2H), 2.74 (t, 2H, J=4.2 Hz), 2.59 (d, 1H, J=6.6 Hz), 1.05-2.48 (m, 27H), 0.95 (s, 3H), 0.70 (s, 3H); ESI-MS (m/z) 527.4 [M+1]⁺.

### Example 17

### Preparation of Compound II1E-03

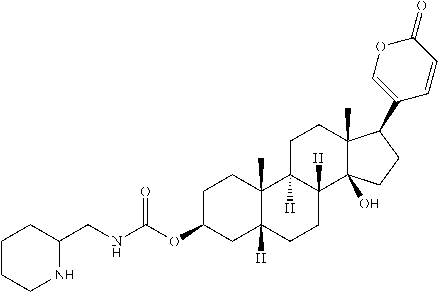

The reaction operations were as the preparation of compound II1B-01, except that 2-aminomethylpiperidine was used instead of N,N-dimethylethylenediamine. The eluent for the silica gel column chromatography is petroleum ether/acetone/ammonia water (50:50:0.5), and the yield is 94%. ¹H NMR (CDCl₃, 300 MHz) δ: 7.83 (dd, 1H, J=9.6, 2.4 Hz), 7.23 (d, 1H, J=2.4 Hz), 6.25 (d, 1H, J=9.6 Hz), 4.98 (br s, 1H), 3.10 (m, 1H), 3.08 (m, 2H), 2.63 (m, 2H), 1.05-2.46 (m, 26H), 0.94 (s, 3H), 0.70 (s, 3H); ESI-MS (m/z) 527.5 [M+1]⁺.

### Example 18

### Preparation of Compound II1E-04

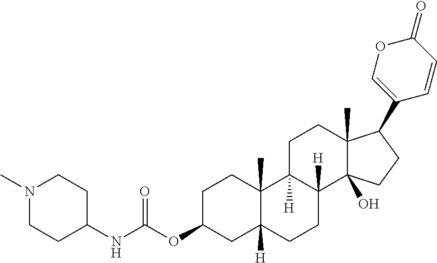

The reaction operations were as the preparation of compound II1B-01, except that 4-amino-N-methylpiperidine was used instead of N,N-dimethylethylenediamine. The eluent for the silica gel column chromatography is petroleum ether/acetone/ammonia water (50:50:0.5), and the yield is 83%. ¹H NMR (CDCl₃, 400 MHz) δ: 7.83 (d, 1H, J=9.7 Hz), 7.23 (s, 1H), 6.26 (d, 1H, J=9.7 Hz), 4.98 (br s, 1H), 3.74 (m, 1H), 3.54 (d, 2H, J=11.2 Hz), 2.80 (m, 5H), 1.06-2.45 (m, 26H), 0.94 (s, 3H), 0.69 (s, 3H); ESI-MS (m/z) 527.3 [M+1]⁺.

### Example 19

### Preparation of Compound II1E-05

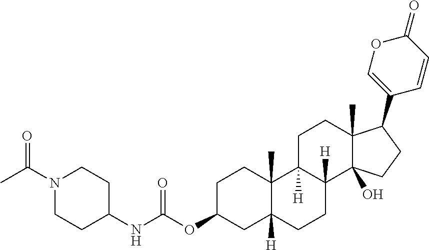

The reaction operations were as the preparation of compound II1B-01, except that 4-amino-1-acetopiperidine was used instead of N,N-dimethylethylenediamine. The eluent for the silica gel column chromatography is petroleum ether/acetone (50:50), and the yield is 81%. ¹H NMR (CDCl₃, 400 MHz) δ: 7.84 (d, 1H, J=9.7 Hz), 7.22 (s, 1H), 6.23 (d, 1H, J=9.7 Hz), 5.67 (d, 1H, J=7.8 Hz), 4.97 (br s, 1H), 4.08 (d, 2H, J=13.8 Hz), 3.91 (m, 1H), 2.88 (m, 4H), 2.45 (m, 1H), 1.20-2.40 (m, 25H), 0.93 (s, 3H), 0.68 (s, 3H); ESI-MS (m/z) 555.2 [M+1]⁺.

### Example 20

### Preparation of Compound II1D-06

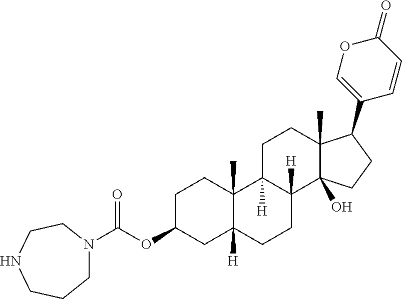

The reaction operations were as the preparation of compound II1B-01, except that homopiperazine was used instead of N,N-dimethylethylenediamine. The eluent for the silica gel column chromatography is petroleum ether/acetone/ammonia water (50:50:0.5), and the yield is 86%. ¹H NMR (CDCl₃, 300 MHz) δ: 7.83 (dd, 1H, J=9.6, 2.4 Hz), 7.23 (d, 1H, J=2.4 Hz), 6.25 (d, 1H, J=9.6 Hz), 5.03 (br s, 1H), 3.52 (m, 4H), 2.93 (t, 2H, J=5.1 Hz), 2.87 (m, 2H), 1.17-2.46 (m, 24H), 0.95 (s, 3H), 0.70 (s, 3H); ¹³C NMR (CDCl₃, 75 MHz) δ: 16.7, 21.6, 21.7, 24.2, 25.6, 26.7, 28.9, 29.9, 30.6, 31.1, 33.0, 35.4, 36.1, 37.5, 41.1, 42.6, 46.0, 48.2, 48.6, 49.5, 49.7, 51.5, 71.1, 85.5, 115.5, 122.9, 147.1, 148.8, 156.0, 162.6; ESI-MS (m/z) 513.3 [M+1]⁺.

### Example 21

### Preparation of Compound II1D-19

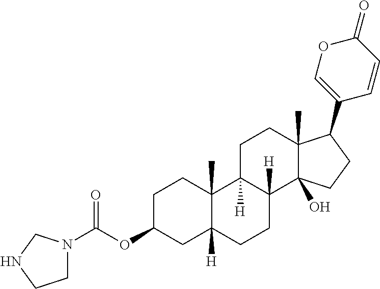

The reaction operations were as the preparation of compound II1B-01, except that imidazoline was used instead of N,N-dimethylethylenediamine. The eluent for the silica gel column chromatography is petroleum ether/acetone/ammonia water (50:50:0.5), and the yield is 43%. ¹H NMR (CDCl₃, 400 MHz) δ: 7.84 (dd, 1H, J=9.7, 3.0 Hz), 7.23 (d, 1H, J=3.0 Hz), 6.27 (d, 1H, J=9.7 Hz), 5.03 (br s, 1H), 4.15 (s, 1H), 4.12 (s, 1H), 3.47 (m, 2H), 3.29 (s, 1H), 2.98 (m, 2H), 2.46 (m, 1H), 1.18-2.22 (m, 21H), 0.95 (s, 3H), 0.70 (s, 3H); ESI-MS (m/z) 485.3 [M+1]⁺.

### Example 22

### Preparation of Compound II1D-20

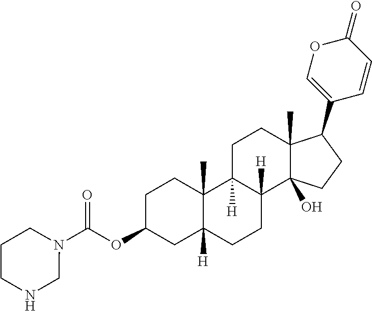

The reaction operations were as the preparation of compound II1B-01, except that hexahydropyrimidine was used instead of N,N-dimethylethylenediamine. The eluent for the silica gel column chromatography is petroleum ether/acetone/ammonia water (50:50:0.5), and the yield is 36%. ¹H NMR (CDCl₃, 400 MHz) δ: 7.84 (d, 1H, J=9.6 Hz), 7.22 (s, 1H), 6.26 (d, 1H, J=9.6 Hz), 5.02 (br s, 1H), 4.28 (s, 2H), 3.61 (t, 1H, J=3.0 Hz), 3.52 (m, 1H), 3.25 (s, 1H), 2.98 (t, 1H, J=3.0 Hz), 2.82 (m, 1H), 2.45 (m, 1H), 1.24-2.24 (m, 23H), 0.95 (s, 3H), 0.70 (s, 3H); ESI-MS (m/z) 485.3 [M+1]⁺.

### Example 23

### Preparation of Compound II1D-22

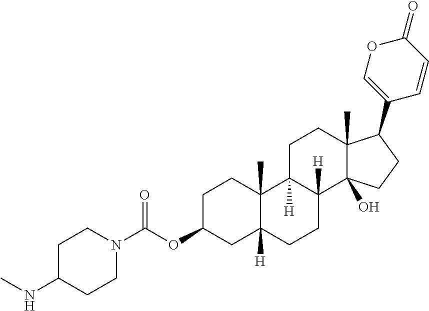

The reaction operations were as the preparation of compound II1B-01, except that piperidine-4-one was used instead of N,N-dimethylethylenediamine, to prepare an intermediate which then reacted with methylamine through a reduction by sodium cyanoborohydride. The eluent for the silica gel column chromatography is petroleum ether/acetone/ammonia water (50:50:0.5), and the yield is 45%. ¹H NMR (CDCl₃, 300 MHz) δ: 7.84 (dd, 1H, J=9.6, 2.7 Hz), 7.22 (d, 1H, J=2.7 Hz), 6.25 (d, 1H, J=9.6 Hz), 4.99 (br s, 1H), 4.07 (br s, 2H), 2.86 (t, 2H, J=10.5 Hz), 2.52 (m, 3H), 2.44 (s, 3H), 2.20 (m, 1H), 1.30-2.20 (m, 26H), 0.94 (s, 3H), 0.69 (s, 3H); ESI-MS (m/z) 527.7 [M+1]⁺.

### Example 24

### Preparation of Compound II1D-23

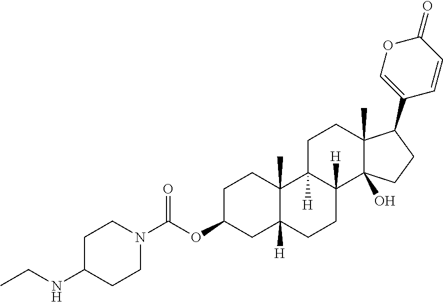

The reaction operations were as the preparation of compound II1B-01, except that piperidine-4-one was used instead of N,N-dimethylethylenediamine, to prepare an intermediate which then reacted with ethylamine through reduction by sodium cyanoborohydride. The eluent for the silica gel column chromatography is petroleum ether/acetone/ammonia water (50:50:0.5), and the yield is 47%. ¹H NMR (CDCl₃, 300 MHz) δ: 7.83 (dd, 1H, J=9.6, 2.7 Hz), 7.25 (d, 1H, J=2.7 Hz), 6.26 (d, 1H, J=9.6 Hz), 5.00 (br s, 1H), 4.10 (br s, 2H), 2.84 (t, 2H, J=10.5 Hz), 2.69 (q, 2H, J=6.00 Hz), 1.25-2.48 (m, 26H), 1.20 (t, 3H, J=6.00 Hz), 0.95 (s, 3H), 0.70 (s, 3H); ESI-MS (m/z) 541.4 [M+1]⁺.

### Example 25

### Preparation of Compound II1D-24

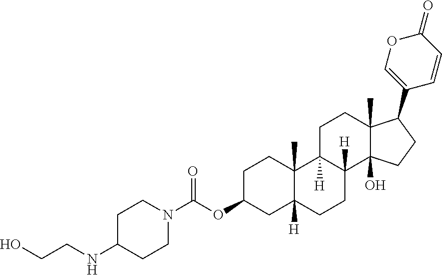

The reaction operations were as the preparation of compound II1B-01, except that piperidine-4-one was used instead of N,N-dimethylethylenediamine, to prepare an intermediate which then reacted with 2-hydroxyethylamine through reduction by sodium cyanoborohydride. The eluent for the silica gel column chromatography is petroleum ether/acetone/ammonia water (50:50:0.5), and the yield is 46%. ¹H NMR (CDCl₃, 300 MHz) δ: 7.83 (dd, 1H, J=9.6, 2.7 Hz), 7.22 (d, 1H, J=2.7 Hz), 6.25 (d, 1H, J=9.6 Hz), 4.99 (br s, 1H), 3.66 (t, 2H, J=6.0 Hz), 2.82 (t, 2H, J=6.0 Hz), 2.65 (m, 1H), 2.44 (m, 1H), 2.20 (m, 1H), 1.02-2.50 (m, 25H), 0.93 (s, 3H), 0.69 (s, 3H); ESI-MS (m/z) 557.4 [M+1]⁺.

### Example 26

### Preparation of Compound II1D-25

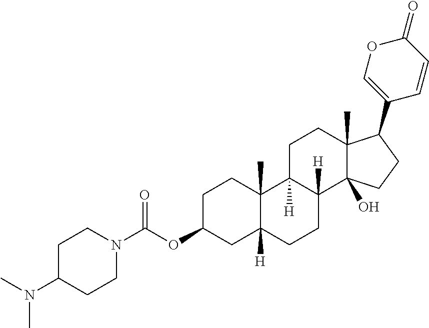

The reaction operations were as the preparation of compound II1B-01, except that piperidine-4-one was used instead of N,N-dimethylethylenediamine, to prepare an intermediate which then reacted with dimethylamine through reduction by sodium cyanoborohydride. The eluent for the silica gel column chromatography is petroleum ether/acetone/ammonia water (50:50:0.5), and the yield is 46%. ¹H NMR (CDCl₃, 300 MHz) δ: 7.83 (dd, 1H, J=9.6, 2.7 Hz), 7.25 (d, 1H, J=2.7 Hz), 6.26 (d, 1H, J=9.6 Hz), 5.01 (br s, 1H), 4.18 (br s, 2H), 2.78 (t, 2H, J=10.5 Hz), 2.45 (m, 1H), 2.29 (s, 6H), 1.15-2.50 (m, 25H), 0.96 (s, 3H), 0.70 (s, 3H); ESI-MS (m/z) 541.4 [M+1]⁺.

### Example 27

### Preparation of Compound II1D-07

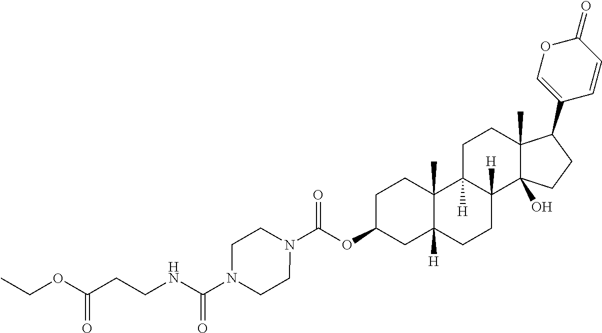

In a 10 mL round bottom flask, compound II1D-05 (30 mg, 0.06 mmol) was dissolved in 2 mL of anhydrous dichloromethane, followed by addition of ethyl 3-isocyanatopropionate (0.18 mmol), and the mixture was stirred at room temperature for 2 hours. After completion of the reaction, the reaction mixture was washed twice with water, dried over anhydrous sodium sulfate, and concentrated under reduced pressure. The residue passed through silica gel column chromatography (chloroform/acetone, 85:15) to give the product II1D-07 with a yield of 98%. ¹H NMR (CDCl₃, 300 MHz) δ: 7.83 (d, 1H, J=9.6 Hz), 7.22 (s, 1H), 6.25 (d, 1H, J=9.6 Hz), 5.26 (t, 1H—N, J=5.4 Hz), 5.03 (br s, 1H), 4.14 (q, 2H, J=6.9 Hz), 3.50 (m, 6H), 3.36 (m, 4H), 2.54 (t, 2H, J=5.7 Hz), 1.26 (t, 3H, J=6.9 Hz), 1.16-2.47 (m, 22H), 0.95 (s, 3H), 0.69 (s, 3H); ESI-MS (m/z) 642.4 [M+1]⁺.

### Example 28

### Preparation of Compound II1D-08

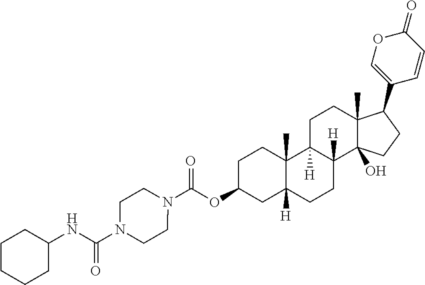

The reaction operations were as the preparation of compound II1D-07, except that cyclohexyl isocyanate was used instead of ethyl 3-isocyanatopropionate. The eluent for the silica gel column chromatography is chloroform/acetone (85:15), and the yield is 83%. ¹H NMR (CDCl₃, 300 MHz) δ: 7.83 (dd, 1H, J=9.9, 2.4 Hz), 7.22 (d, 1H, J=2.4 Hz), 6.25 (d, 1H, J=9.9 Hz), 5.03 (br s, 1H), 4.26 (d, 1H—N, J=7.2 Hz), 3.63 (m, 1H), 3.47 (m, 4H), 3.35 (m, 4H), 1.02-2.48 (m, 32H), 0.95 (s, 3H), 0.69 (s, 3H); ESI-MS (m/z) 624.3 [M+1]⁺.

### Example 29

### Preparation of Compound II1D-09

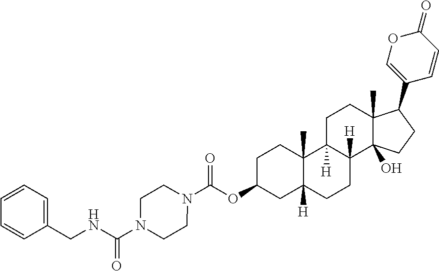

The reaction operations were as the preparation of compound II1D-07, except that benzyl isocyanate was used instead of ethyl 3-isocyanatopropionate. The eluent for the silica gel column chromatography is chloroform/acetone (85:15), and the yield is 92%. ¹H NMR (CDCl₃, 300 MHz) δ: 7.83 (d, 1H, J=9.9 Hz), 7.32 (m, 5H), 7.22 (s, 1H), 6.24 (d, 1H, J=9.9 Hz), 5.03 (br s, 1H), 4.76 (br s, 1H—N), 4.22 (d, 2H, J=5.4 Hz), 3.49 (m, 4H), 3.40 (m, 4H), 1.16-2.48 (m, 22H), 0.94 (s, 3H), 0.69 (s, 3H); ESI-MS (m/z) 632.5 [M+1]⁺.

### Example 30

### Preparation of Compound II1D-10

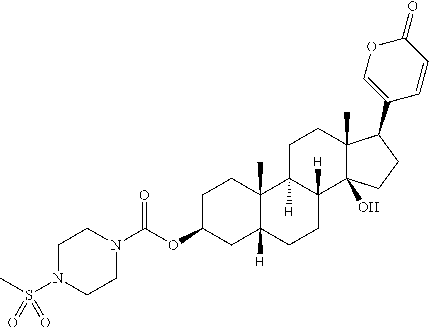

In a 10 mL round bottom flask, compound II1D-05 (30 mg, 0.06 mmol) was dissolved in 2 mL of anhydrous dichloromethane, followed by addition of triethylamine (25 μL) and methanesulfonyl chloride (0.18 mmol) in sequence, and the mixture was stirred at room temperature for 2 hours. After completion of the reaction, the reaction mixture was washed twice with water, dried over anhydrous sodium sulfate, and concentrated under reduced pressure. The residue passed through silica gel column chromatography (chloroform/acetone, 80:20) to give the compound II1D-10 with a yield of 95%. ¹H NMR (CDCl₃, 300 MHz) δ: 7.83 (d, 1H, J=9.9 Hz), 7.23 (s, 1H), 6.26 (d, 1H, J=9.9 Hz), 5.05 (br s, 1H), 3.60 (t, 4H, J=4.5 Hz), 3.22 (t, 4H, J=4.5 Hz), 2.80 (s, 3H), 1.20-2.49 (m, 22H), 0.94 (s, 3H), 0.69 (s, 3H); ESI-MS (m/z) 577.2 [M+1]⁺.

### Example 31

### Preparation of Compound II1D-11

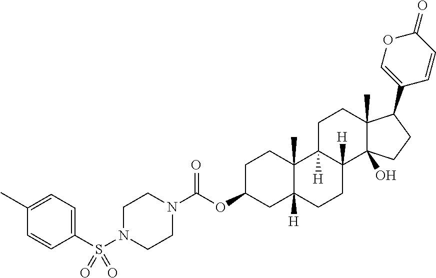

The reaction operations were as the preparation of compound II1D-10, except that p-methylbenzenesulfonyl chloride was used instead of methanesulfonyl chloride. The eluent for the silica gel column chromatography is petroleum ether/acetone (80:20), and the yield is 92%. ¹H NMR (CDCl₃, 300 MHz) δ: 7.82 (d, 1H, J=9.6 Hz), 7.62 (d, 2H, J=8.1 Hz), 7.33 (d, 2H, J=8.1 Hz), 7.22 (s, 1H), 6.24 (d, 1H, J=9.6 Hz), 4.95 (br s, 1H), 3.55 (t, 4H, J=5.1 Hz), 2.97 (t, 4H, J=5.1 Hz), 2.43 (s, 3H), 1.20-2.49 (m, 22H), 0.92 (s, 3H), 0.68 (s, 3H); ESI-MS (m/z) 653.4 [M+1]⁺.

### Example 32

### Preparation of Compound II1D-12

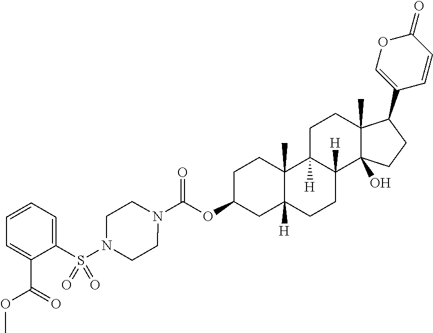

The reaction operations were as the preparation of compound II1D-10, except that methyl 2-(chlorosulfonyl)benzoate was used instead of methanesulfonyl chloride. The eluent for the silica gel column chromatography is petroleum ether/acetone (70:30), and the yield is 93%. ¹H NMR (CDCl₃, 300 MHz) δ: 7.83 (m, 2H), 7.62 (m, 2H), 7.49 (d, 1H, J=9.6 Hz), 7.22 (s, 1H), 6.25 (d, 1H, J=9.6 Hz), 4.98 (br s, 1H), 3.94 (s, 3H), 3.55 (t, 4H, J=5.1 Hz), 3.19 (t, 4H, J=5.1 Hz), 1.20-2.49 (m, 22H), 0.94 (s, 3H), 0.69 (s, 3H); ESI-MS (m/z) 697.3 [M+1]⁺.

### Example 33

### Preparation of Compound II1D-13

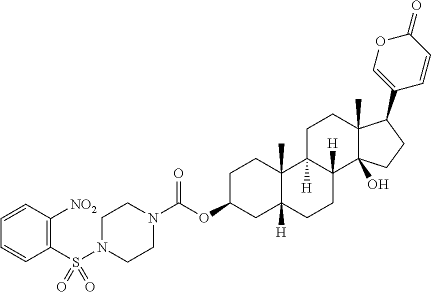

The reaction operations were as the preparation of compound II1D-10, except that o-nitrobenzenesulfonyl chloride was used instead of methanesulfonyl chloride. The eluent for the silica gel column chromatography is petroleum ether/acetone (70:30), and the yield is 90%. ¹H NMR (CDCl₃, 300 MHz) δ: 7.99 (dd, 1H, J=7.2, 1.8 Hz), 7.83 (d, 1H, J=9.6 Hz), 7.74 (m, 2H), 7.63 (dd, 1H, J=7.2, 1.8 Hz), 7.26 (s, 1H), 6.25 (d, 1H, J=9.6 Hz), 5.01 (br s, 1H), 3.57 (t, 4H, J=5.1 Hz), 3.30 (t, 4H, J=5.1 Hz), 1.20-2.49 (m, 22H), 0.95 (s, 3H), 0.69 (s, 3H); ESI-MS (m/z) 684.7 [M+1]⁺.

### Example 34

### Preparation of Compound II1D-14

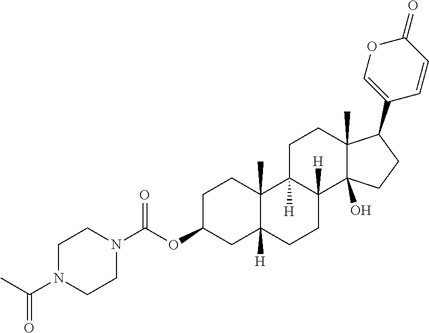

In a 10 mL round bottom flask, compound II1D-05 (30 mg, 0.06 mmol) was dissolved in 2 mL of anhydrous dichloromethane, followed by addition of pyridine (15 μL) and the corresponding acetic anhydride (0.18 mmol) in sequence, and the mixture was stirred at room temperature for 2 hours. After completion of the reaction, the reaction mixture was washed twice with water, dried over anhydrous sodium sulfate, and concentrated under reduced pressure. The residue passed through silica gel column chromatography (petroleum ether/acetone, 70:30) to give the compound II1D-14 with a yield of 83%. ¹H NMR (CDCl₃, 300 MHz) δ: 7.83 (d, 1H, J=9.9 Hz), 7.23 (s, 1H), 6.25 (d, 1H, J=9.9 Hz), 5.04 (br s, 1H), 3.60 (t, 2H, J=5.1 Hz), 3.47 (t, 6H, J=5.1 Hz), 2.11 (s, 3H), 1.20-2.49 (m, 22H), 0.95 (s, 3H), 0.69 (s, 3H); ESI-MS (m/z) 541.2 [M+1]⁺.

### Example 35

### Preparation of Compound II1D-15

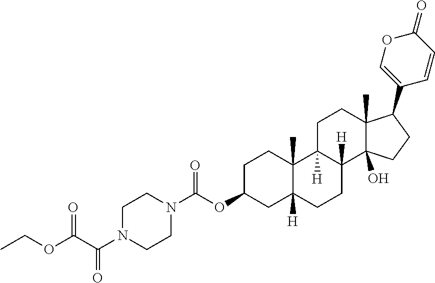

The reaction operations were as the preparation of compound II1D-14, except that ethyl oxalyl monochloride was used instead of acetic anhydride. The eluent for the silica gel column chromatography is petroleum ether/acetone (70:30), and the yield is 89%. ¹H NMR (CDCl₃, 300 MHz) δ: 7.83 (d, 1H, J=9.6 Hz), 7.23 (s, 1H), 6.26 (d, 1H, J=9.6 Hz), 5.05 (br s, 1H), 4.35 (q, 2H, J=7.2 Hz), 3.63 (t, 2H, J=5.1 Hz), 3.54 (t, 4H, J=5.1 Hz), 3.45 (t, 2H, J=5.1 Hz), 1.37 (t, 3H, J=7.2 Hz), 1.20-2.49 (m, 22H), 0.96 (s, 3H), 0.70 (s, 3H); ESI-MS (m/z) 599.3 [M+1]⁺.

### Example 36

### Preparation of Compound II1D-16

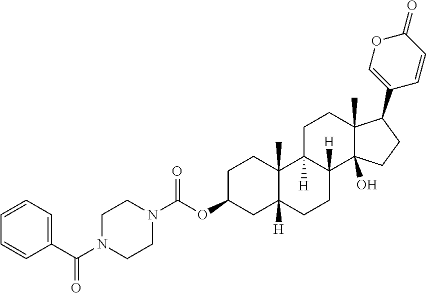

The reaction operations were as the preparation of compound II1D-14, except that benzoyl chloride was used instead of acetic anhydride. The eluent for the silica gel column chromatography is petroleum ether/acetone (70:30), and the yield is 86%. ¹H NMR (CDCl₃, 300 MHz) δ: 8.08 (d, 1H, J=9.9 Hz), 7.58 (m, 5H), 7.23 (s, 1H), 6.26 (d, 1H, J=9.9 Hz), 5.05 (br s, 1H), 3.76 (m, 2H), 3.50 (m, 6H), 1.20-2.49 (m, 22H), 0.94 (s, 3H), 0.69 (s, 3H); ESI-MS (m/z) 603.4 [M+1]⁺.

### Example 37

### Preparation of Compound III1A-01

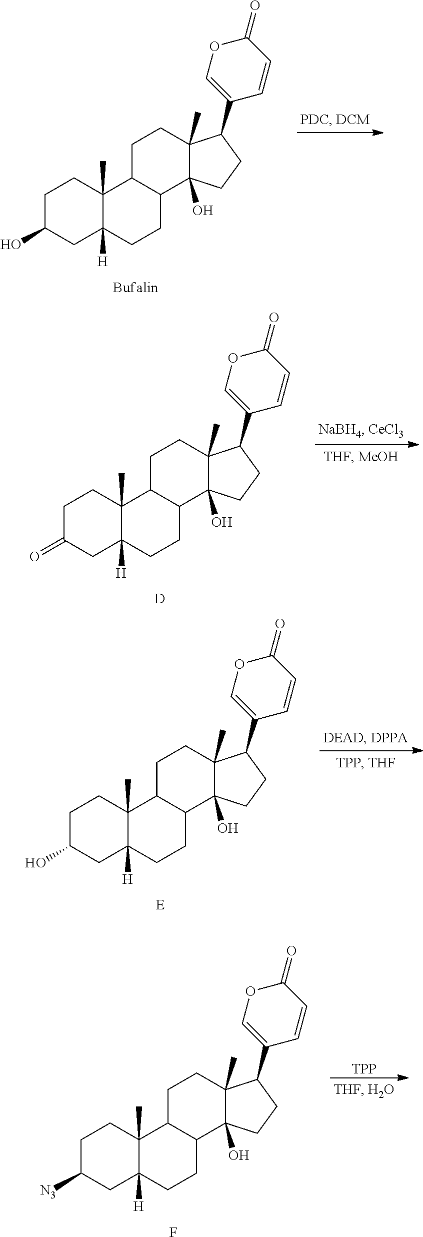

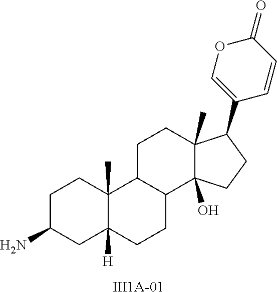

Bufalin (1 mmol) was dissolved with dichloromethane (DCM) in a 25 mL round bottom flask at room temperature, followed by slow addition of pyridinium dichromate (PDC) (4 mmol). The mixture was stirred overnight, and filtered to give a reddish brown liquid. The liquid was then concentrated under reduced pressure to remove the solvent, and the residue passed through silica gel column chromatography (petroleum ether:acetone=5:1) to give a white solid D.

Cerium trichloride heptahydrate (1.3 mmol) was dissolved with methanol in a 25 mL round bottom flask at 0° C., followed by slow addition of sodium borohydride (NaBH₄)(1.2 mmol). The temperature of the reaction system was decreased to −78° C., and a solution of the above compound D (1 mmol) in tetrahydrofuran (10 mL) was added slowly to react for 1 hour. 1 mL of water was introduced dropwisely, and the temperature of the solution was raised to room temperature, and then the system was distilled under reduced pressure to remove the solvent, dissolved with dichloromethane, and washed respectively with water and saturated saline. The organic phase was distilled under reduced pressure to remove the solvent and then give a white solid E, which was directly used in the next step.

The above white solid product E (1 mmol) and triphenylphosphine (TPP, 2 mmol) were dissolved with anhydrous tetrahydrofuran (10 mL) under icebath under nitrogen atmosphere, followed by addition of diphenylphosphoryl azide (DPPA) and then slow addition of diethyl azodicarboxylate (DEAD). The solution turned to yellow gradually. The temperature of the reaction system was raised to room temperature, and then the system was stirred overnight, distilled under reduced pressure to remove the solvent, dissolved with dichloromethane, and washed respectively with water and saturated saline. The organic phase was collected, and distilled under reduced pressure to give a yellow oil, which was passed through silica gel column chromatography (petroleum ether:acetone=6:1) to afford a white solid F.

The above solid F (1 mmol) and triphenylphosphine (1.2 mmol) were dissolved with tetrahydrofuran (10 mL), followed by addition of 1 mL of water, and then the system was refluxed overnight at 70° C. and distilled under reduced pressure to remove the solvent. The residue was dissolved with dichloromethane, and washed respectively with water and saturated saline. The organic phase was collected, and distilled under reduced pressure to give a yellow oil, which was passed through silica gel column chromatography eluting with petroleum ether/acetone (4:1) and then with petroleum ether/acetone/ammonia water (20:10:0.1) to afford a white solid III1A-01. ¹H NMR (CDCl₃, 300 MHz) δ: 7.85 (dd, 1H, J=9.6, 2.7 Hz), 7.23 (d, 1H, J=2.7 Hz), 6.27 (d, 1H, J=9.6 Hz), 5.30 (br s, 1H), 3.48 (br s, 1H), 3.27 (br s, 1H), 2.45 (d, 1H, J=2.7 Hz), 1.20-2.49 (m, 21H), 0.95 (s, 3H), 0.70 (s, 3H); ESI-MS (m/z) 386.4 [M+1]⁺.

### Example 38

### Preparation of Compound III1D-02

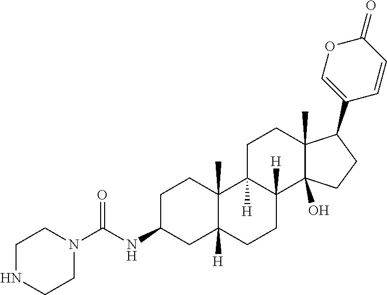

Compound III1A-01 (1 mmol) was dissolved with dichloromethane in a 10 mL round bottom flask at room temperature, followed by addition of carbonyldiimidazole (3 mmol) and triethylamine (3 mmol) to react for 2 hours. The reaction solution was washed with water and dried, followed by addition of piperazine (3 mmol) and triethylamine (3 mmol) to react for 3 hours. After completion of the reaction, the reaction solution was washed with water and dried, and the residue was passed through silica gel column chromatography (petroleum ether/acetone/ammonia water (50:50:0.5) to afford the product with a yield of 60%. ¹H NMR (CDCl₃, 300 MHz) δ: 7.83 (dd, 1H, J=9.6, 2.4 Hz), 7.23 (d, 1H, J=2.4 Hz), 6.26 (d, 1H, J=9.6 Hz), 4.65 (d, 1H, J=6.0 Hz), 4.12 (s, 1H), 3.34 (t, 4H, J=5.1 Hz), 2.88 (t, 4H, J=5.1 Hz), 2.46 (m, 1H), 1.04-2.40 (m, 21H), 0.95 (s, 3H), 0.70 (s, 3H); ESI-MS (m/z) 498.3 [M+1]⁺.

### Example 39

### Preparation of Compound III1D-03

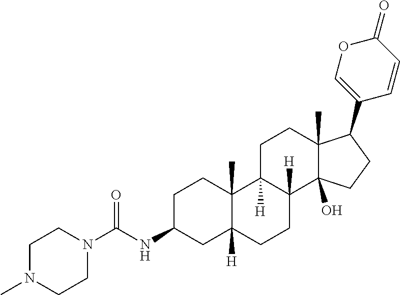

The reaction operations were as the preparation of compound III1D-02, except that N-methylpiperazine was used instead of piperazine. The eluent for the silica gel column chromatography is petroleum ether/acetone/ammonia water (50:50:0.5), and the yield is 53%. ¹H NMR (CDCl₃, 300 MHz) δ: 7.83 (dd, 1H, J=9.6, 2.7 Hz), 7.23 (d, 1H, J=2.7 Hz), 6.25 (d, 1H, J=9.6 Hz), 4.61 (d, 1H, J=6.0 Hz), 4.12 (s, 1H), 3.49 (t, 4H, J=4.8 Hz), 2.39 (t, 4H, J=4.8 Hz), 2.30 (s, 3H), 1.12-2.27 (m, 22H), 0.95 (s, 3H), 0.70 (s, 3H); ESI-MS (m/z) 512.3 [M+1]+.

### Example 40

### Preparation of Compound III1D-04

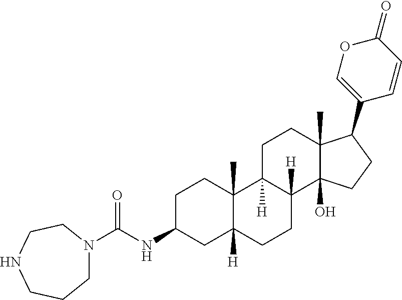

The reaction operations were as the preparation of compound III1D-02, except that homopiperazine was used instead of piperazine. The eluent for the silica gel column chromatography is petroleum ether/acetone/ammonia water (50:50:0.5), and the yield is 56%. ¹H NMR (CDCl₃, 300 MHz) δ: 7.83 (dd, 1H, J=9.6, 2.4 Hz), 7.23 (d, 1H, J=2.4 Hz), 6.26 (d, 1H, J=9.6 Hz), 4.61 (d, 1H, J=6.0 Hz), 4.12 (s, 1H), 3.50 (m, 4H), 2.96 (t, 2H, J=6.0 Hz), 2.90 (t, 2H, J=6.0 Hz), 2.45 (m, 1H), 1.12-2.44 (m, 23H), 0.95 (s, 3H), 0.69 (s, 3H); ESI-MS (m/z) 512.4 [M+1]⁺.

### Example 41

### Preparation of Compound III1E-01

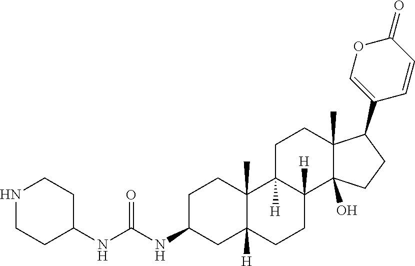

The reaction operations were as the preparation of compound III1D-02, except that 4-aminopiperidine was used instead of piperazine. The eluent for the silica gel column chromatography is petroleum ether/acetone/ammonia water (50:50:0.5), and the yield is 56%. ¹H NMR (CDCl₃, 300 MHz) δ: 7.84 (dd, 1H, J=9.6, 2.7 Hz), 7.23 (d, 1H, J=2.7 Hz), 6.25 (d, 1H, J=9.6 Hz), 4.68 (d, 1H, J=6.0 Hz), 4.10 (br s, 1H), 3.86 (d, 2H, J=9.0 Hz), 2.83 (t, 4H, J=12.0 Hz), 2.46 (m, 1H), 1.12-2.45 (m, 25H), 0.93 (s, 3H), 0.73 (s, 3H); ESI-MS (m/z) 512.2 [M+1]⁺.

### Example 42

### Preparation of Compound 1111E-02

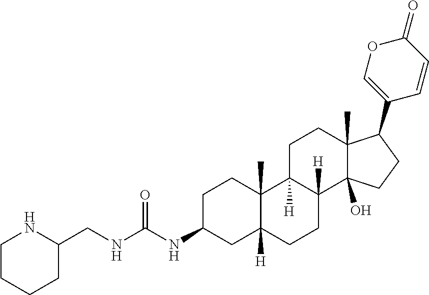

The reaction operations were as the preparation of compound III1D-02, except that 2-aminomethylpiperidine was used instead of piperazine. The eluent for the silica gel column chromatography is petroleum ether/acetone/ammonia water (50:50:0.5), and the yield is 53%. ¹H NMR (CDCl₃, 300 MHz) δ: 7.83 (dd, 1H, J=9.6, 2.7 Hz), 7.23 (d, 1H, J=2.7 Hz), 6.25 (d, 1H, J=9.6 Hz), 4.61 (d, 1H, J=6.0 Hz), 4.12 (s, 1H), 3.49 (d, 2H, J=4.8 Hz), 3.12 (m, 1H), 2.39 (m, 2H), 1.12-2.30 (m, 27H), 0.95 (s, 3H), 0.70 (s, 3H); ESI-MS (m/z) 526.3 [M+1]⁺.

### Example 43

### Preparation of the hydrochloride salt of compound II1B-02 (II1B-02.HCl)

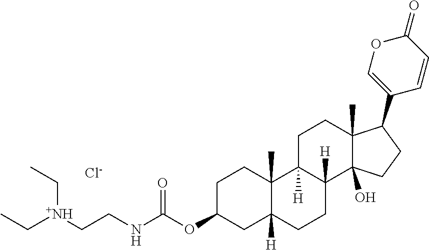

II1B-02 (1 mmol) was dissolved in 30 mL of 1% diluted hydrochloric acid, followed by stirring for 2 hours at room temperature. After completion of the reaction, a crude product was obtained through filtration, and recrystallized from ethanol to afford a white solid II1B-02.HCl with a yield of 80%.

All the hydrochloride salts of other compounds may be prepared by reaction of the corresponding compound with a diluted hydrochloric acid according to the process of Example 43.

The organic or inorganic acid salt of the compound described in the present invention may be prepared by reaction of the compound with the corresponding organic or inorganic acid according to a similar process as that of Example 43.

### Experimental Example

### Experimental Example 1

### In Vitro Antitumor Activity Assay

### 1) Experimental Materials

HeLa human cervical carcinoma cell line, A-549 human non-small cell lung carcinoma cell line, NCI-H2228 human lung carcinoma cell line, NCI-H460 human lung carcinoma cell line, MDA-MB-231 human breast carcinoma cell line, MCF-7 human breast carcinoma cell line, Bel-7402 human hepatic carcinoma cell line, Hep3B human hepatic carcinoma cell line, QGY-7703 human hepatic carcinoma cell line, MV-4-11 human leukemic cell line, DAUDI human leukemic cell line, Jurkat human leukemic cell line, A498 renal carcinoma cell line, LoVo human colon carcinoma cell line, HCT1116 human colon carcinoma cell line, A431 human skin carcinoma cell line, PANC-1 human pancreatic carcinoma cell line, U87-MG human brain carcinoma cell line, SH-SY5Y human brain carcinoma cell line, RPMI-8226 human bone carcinoma cell line, HT1080 human fibrosarcoma cell line, PC-3 human prostatic carcinoma cell line, AGS human gastric adenocarcinoma cell line, and BGC-823 human gastric adenocarcinoma cell line were purchased from the Cell Bank of the Chinese Academy of Sciences.

The positive control is bufalin (formulated according to the common method), its purity is 98% or more by HPLC-UV and its structure is identified by NMR. The compounds to be tested and the positive control were diluted with normal saline with a concentration gradient of 10⁻⁴ M, 10⁻⁵ M, 10⁻⁶ M, 10⁻⁷ M and 10⁻⁸ M.

### 2) Experimental Protocol

SRB Reduction Method:

According to cell growth rate, tumor cells in the log phase of growth were seeded into 96-well culture plates at 100 μL per well, and allowed to attach for 24 hours, followed by addition of a test compound or positive control at 10 μL per well. For each concentration, the test was carried out in triplicate wells. Solvent control wells with addition of normal saline into cell culture and a blank well containing only cell culture medium but without cells for zeroing were also included. The tumor cells were incubated for 72 hours at 37° C. under 5% CO₂, and then the culture medium (RPMI-1640) was removed. The cells were fixed with 10% cool TCA by incubating for 1 h at 4° C., washed with distilled water for 5 times, and dried naturally in the air. A solution of SRB (Sigma) (4 mg/mL) in 1% glacial acetic acid was added at 100 μL per well, and the cells were stained for 15 minutes at room temperature, and the supernatant was discarded. The plates were washed for 5 times with 1% acetic acid, and dried in air. Finally, Tris solution was added at 150 μL per well, and the absorbance (A) was measured at a wavelength of 515 nm using a microplate reader. The growth inhibition on tumor cell proliferation was calculated according to the following equation:

Growth Inhibition (%)=[(Absorbance of negative control−Absorbance of blank)−(Absorbance of sample−Absorbance of blank)]/(Absorbance of negative control−Absorbance of blank)×100%

Drug concentration: 10 μM, 1 μM, 0.1 μM, 10 nM, 1 nM, 0.1 nM.

IC₅₀ was fitted by GraphPad Prism 4.

The inhibitory activities of the derivatives prepared by us on cell proliferation were first evaluated on human-derived HeLa tumor cell line. The results are listed in Table 1.

TABLE 1
The inhibitory activities of some bufalin derivatives
on cell proliferation of human-derived HeLa cell line

| Compound | IC₅₀ (nM) |
|---|---|
| bufalin | 7.27 |
| II1B-01 | 1.79 |
| II1B-02 | 3.73 |
| II1B-03 | 1.58 |
| II1B-04 | 2.17 |
| II1B-05 | 1.65 |
| II1B-06 | 1.11 |
| II1B-07 | 1.15 |
| II1B-08 | 3.43 |
| II1B-09 | 5.72 |
| II1B-10 | 1.82 |
| II1D-03 | 3.49 |
| II1D-04 | 37.97 |
| II1D-06 | 0.45 |
| II1D-07 | 2.60 |
| II1D-10 | 16.27 |
| II1D-12 | 41.75 |
| II1D-14 | 23.13 |
| II1D-16 | 44.65 |
| II1D-19 | 5.80 |
| II1D-20 | 1.19 |
| II1D-22 | 7.34 |
| II1D-23 | 1.12 |
| II1D-24 | 7.30 |
| II1D-25 | 5.44 |
| II1E-01 | 1.40 |
| II1E-02 | 4.31 |
| II1E-03 | 0.80 |
| III1D-02 | 5.68 |
| III1D-03 | 41.62 |
| III1D-04 | 3.98 |
| III1E-01 | 7.20 |
| III1E-02 | 8.68 |

Through evaluation on the inhibitory activities of bufalin derivatives on cell proliferation of human-derived HeLa tumor cell line, one could find that some bufalin derivatives have higher inhibitory activities on cell proliferation of human-derived HeLa tumor cell line than bufalin.

Some bufalin derivatives with higher activities were selected to evaluate the inhibitory activities on cell proliferation of several human-derived tumor cell lines. The results are listed in Table 2.

TABLE 2
The inhibitory activities of some bufalin derivatives on cell
proliferation of several human-derived tumor cell lines

| | IC₅₀ (nM) | | | |
|---|---|---|---|---|
| Compound | A549 | MCF-7 | PC-3 | LoVo |
| bufalin | 6.53 ± 1.64 | 28.78 ± 3.71 | 17.31 ± 2.76 | 29.53 ± 1.00 |
| II1B-02 | 0.39 ± 0.08 | 18.77 ± 2.68 | 20.06 ± 4.08 | 15.66 ± 1.67 |
| II1B-03 | 2.81 ± 0.43 | 10.64 ± 3.04 | 27.30 ± 2.46 | 13.36 ± 3.37 |
| II1B-05 | 2.83 ± 0.90 | 7.98 ± 1.90 | 12.63 ± 0.86 | 14.45 ± 0.76 |
| II1D-06 | 0.87 ± 0.02 | 2.13 ± 0.29 | 1.25 ± 0.46 | 0.58 ± 0.29 |
| II1E-03 | 0.58 ± 0.17 | 30.96 ± 3.07 | 14.79 ± 1.12 | 23.41 ± 3.68 |

The above experimental data demonstrate that some bufalin derivatives have significantly improved inhibitory activities on cell proliferation of various human-derived tumor cell lines.

The inhibitory activities on cell proliferation of 17 human-derived tumor cell lines were evaluated for bufalin, II1E-01 and II1E-01.HCl.

TABLE 3
The inhibitory activities of bufalin, II1E-01 and II1E-01•HCl
on cell proliferation of human-derived tumor cell lines

| Compound | IC₅₀ (nM) (72 h) | | | | |
|---|---|---|---|---|---|
| | MV-4-11 | Jurkat | Daudi | PC-3 | HT1080 |
| bufalin | 6.26 ± 2.22 | 8.79 ± 2.48 | 12.56 ± 0.76 | 5.00 ± 0.45 | 11.31 ± 0.38 |
| II1E-01 | 1.12 ± 0.29 | 1.09 ± 0.26 | 2.86 ± 0.21 | 0.41 ± 0.10 | 2.90 ± 0.33 |
| II1E-01•HCl | 0.64 ± 0.11 | 0.67 ± 0.10 | 2.34 ± 0.22 | 0.30 ± 0.08 | 2.34 ± 0.10 |
| | A549 | NCI-H460 | NCI-H2228 | AGS | BGC-823 |
| bufalin | 8.74 ± 2.52 | 6.89 ± 0.87 | 16.06 ± 2.51 | 51.80 ± 1.34 | 9.06 ± 0.37 |
| II1E-01 | 0.77 ± 0.28 | 0.56 ± 0.05 | 2.50 ± 0.34 | 9.62 ± 0.69 | 0.81 ± 0.07 |
| II1E-01•HCl | 0.40 ± 0.22 | 0.67 ± 0.03 | 1.62 ± 0.50 | 9.35 ± 0.53 | 0.56 ± 0.07 |
| | MDA-MB-231 | MCF-7 | HepG2 | Hep3B | QGY7703 |
| bufalin | 28.73 ± 1.66 | 45.87 ± 5.18 | 6.02 ± 0.71 | >1000 | 12.77 ± 0.47 |
| II1E-01 | 7.46 ± 0.33 | 9.97 ± 1.45 | 0.51 ± 0.04 | >1000 | 1.11 ± 0.08 |
| II1E-01•HCl | 7.31 ± 0.21 | 10.78 ± 1.08 | 0.33 ± 0.02 | >1000 | 1.72 ± 0.08 |
| | A498 | HCT-116 | A431 | PANC-1 | RPMI8226 |
| bufalin | 6.41 ± 0.75 | 9.69 ± 0.64 | 26.61 ± 5.35 | 9.88 ± 0.27 | 13.50 ± 0.82 |
| II1E-01 | 0.40 ± 0.03 | 2.91 ± 0.09 | 2.62 ± 0.19 | 0.72 ± 0.19 | 1.53 ± 0.10 |
| II1E-01•HCl | 0.37 ± 0.05 | 2.13 ± 0.28 | 3.31 ± 0.09 | 0.57 ± 0.06 | 1.71 ± 0.03 |
| | U87-MG | | | | |
| bufalin | 13.04 ± 1.74 | | | | |
| II1E-01 | 1.28 ± 0.36 | | | | |
| II1E-01•HCl | 1.98 ± 0.30 | | | | |

The above experimental data demonstrate that the bufalin derivatives II1E-01 and II1E-01.HCl have significantly improved inhibitory activities on cell proliferation of the selected 16 human-derived tumor cell lines (except Hep3B).

### Experimental Example 2

### In Vivo Acute Toxicity Assay of Compound II1E-01.HCl in Mouse

### 1) Sample to be Tested

Sample Name: bufalin, II1E-01.HCl

### 2) Experimental Protocol

35.8 mg of II1E-01.HCl was weighted, added with 1.78 mL of anhydrous ethanol, and dissolved completely under ultrasonication. 16.02 mL of 5% glucose injection was added and agitated homogeneously. The solution was filtered with a 0.2 μm filtration membrane to obtain a stock solution with a concentration of 2 mg/mL, wherein the content of ethanol is 10%. The highest dosage is 20 mg/kg, calculated based on the administration volume of 0.1 mL/10 g body weight of mouse.

### 3) Experimental Results

The experimental results indicated that, for compound II1E-01.HCl, LD₅₀ of intravenous administration was about 13.60 mg/kg for male mouse, and about 16.51 mg/kg for female mouse; LD₅₀ of intraperitoneal administration was about 14.75 mg/kg for male mouse, and about 18.21 mg/kg for female mouse; and for bufalin, LD₅₀ of intraperitoneal administration was about 2.4 mg/kg for male mouse, and about 2.8 mg/kg for female mouse. The above experimental results suggest that compound II1E-01.HCl has a lower in vivo acute toxicity in mouse than bufalin.

### Experimental Example 3

### In Vivo Therapeutic Effect Assay Against Human-Derived Lung Carcinoma A549 Cell Xenograft in Nude Mice

### 1) Experimental Object

This experiment is to evaluate the in vivo therapeutic effects of compounds bufalin, II1D-06.HCl, II1E-01.HCl and II1B-02.HCl against human-derived lung carcinoma A549 cell xenograft in nude mice.

### 2) Materials and Protocol

Solvent control: 4% DMSO & 2% Tween-80 & 5% PEG-400 solution in normal saline

1) DMSO: SIGMA-ALDRICH CHEMIE GMBH. 2) Tween-80: SIGMA-ALDRICH CHEMIE GMBH. 3) PEG-400: Nanjing Weier Chemical Ltd. 4) Normal saline: Shanghai Changzheng Fumin Pharmaceutical (Middle China) Ltd.

Compounds to be tested: bufalin, II1B-02.HCl, II1E-01.HCl, II1D-06.HCl

Formulating method: the compound was dissolved with DMSO into a stock solution, which was then diluted into an injection with a final DMSO concentration of 4%.

Positive control: Rapamycin Formulating method: it was formulated into an injection with a final DMSO concentration of 4%.

### 3) Experimental Protocol and Results

After the tumor size reaches about 200 mm³, the mice bearing the tumor were grouped randomly into 8 groups according to the tumor size, including the groups of solvent control, positive control rapamycin 5 mg/kg, II1E-01.HCl 2 mg/kg, II1E-01.HCl 4 mg/kg, II1E-01.HCl 6 mg/kg, II1B-02.HCl 4 mg/kg, II1D-06.HCl 4 mg/kg, bufalin 1.6 mg/kg, wherein the dosage selection is based on that the maximum tolerable dosage is 1.6 mg/kg for bufalin and 6 mg/kg for II1E-01.HCl. Rapamycin was administered intravenously once a week (QW) and the other groups were administered intraperitoneally once every other day (QOD). The tumor was weighted after 21 days to calculate the inhibition rate. The equation for calculating inhibition rate (IR) is IR=(W_{C}−W_{T})/W_{C}×100%, wherein, W_{C} represents the tumor weight of the control group, and W_{T} represents the tumor weight of the treating group. The results are listed in Table 4.

TABLE 4
Therapeutic effects against human non-small cell
lung carcinoma A549 xenograft in nude mice

| | Dosage | Administering | Animal Number | Tumor Weight(g) | Inhibition |
|---|---|---|---|---|---|
| Group | (mg/kg) | method | Beginning/End | Mean ± SE | rate (%) |
| Solvent Control | Solvent | ip | 15/15 | 1.6833 ± 0.0564 | — |
| rapamycin | 5 | iv | 6/6 | 0.6925 ± 0.0652** | 58.86% |
| II1E-01•HCl | 2 | ip | 10/10 | 1.1375 ± 0.1044** | 32.43% |
| II1E-01•HCl | 4 | ip | 10/10 | 0.4953 ± 0.0301** | 70.57% |
| II1E-01•HCl | 6 | ip | 10/10 | 0.2070 ± 0.0193** | 87.70% |
| II1D-06•HCl | 4 | ip | 6/6 | 1.0580 ± 0.0866* | 37.15% |
| II1B-02•HCl | 4 | ip | 6/6 | 0.7013 ± 0.0707* | 58.34% |
| bufalin | 1.6 | ip | 6/6 | 1.5320 ± 0.0624* | 8.99% |

Remarks: comparing with the solvent control group,
*P < 0.05,
**P < 0.01;
ip: Intraperitoneal administration;
iv: Intravenous administration;

II1B-02.HCl and II1E-01.HCl can significantly inhibit the growth of human lung carcinoma A549 xenograft, and II1E-01.HCl has the strongest inhibitory activity and the inhibitory activity is dose-dependent. Bufalin did not express strong inhibitory activity at the maximum tolerable dosage (1.6 mg/kg).

### Experimental Example 4

### Therapeutic Efficiency Assay of II1E-01.HCl Against Human-Derived Hepatic Carcinoma HepG2 cell, Human-Derived Breast Carcinoma MDA-MB-231 Cell, Human-Derived Lung Carcinoma H460 Cell, Human-Derived Colon Carcinoma HCT-116 Cell, Human-Derived Lymphoma MV-4-11 Cell Xenografts in Nude Mice

### 1) Experimental Object

This experiment is to investigate the in vivo inhibitory effect of II1E-01.HCl on growth of human-derived hepatic carcinoma HepG2 cell, human-derived breast carcinoma MDA-MB-231 cell, human-derived lung carcinoma H460 cell, human-derived colon carcinoma HCT-116 cell, human-derived lymphoma MV-4-11 cell xenografts in nude mice.

### 2) Experimental Materials

Formulation of solvent control (5% anhydrous ethanol & 5% aqueous glucose solution): 2.5 mL anhydrous ethanol was added into a 50 mL tube, followed by addition of 47.5 mL 5% glucose solution for injection followed by shaking, and stored at room temperature before use.

Formulation of the preparations of the compounds to be tested: formulations with concentrations of 0.4 mg/mL and 0.6 mg/mL were prepared respectively. An appropriate amount of II1E-01.HCl was weighted and put into a 50 mL tube, followed by addition of corresponding volume of solvent to get the desired concentration and mixed using a vortex mixer. After the solid was dissolved completely, the solution was subpackaged into 4 mL vials and stored at refrigerated condition (2-8° C.) until use.

Positive controls: Vinorelbine, Rapamycin, Sunitinib.

Solvent materials: Tween-80, PEG-400, anhydrous ethanol, 0.5% glucose injection, normal saline.

Experimental animals: Balb/c nude mice (male, six weeks old).

Implanted tumor cell lines: human-derived hepatic carcinoma HepG2 cell, human-derived breast carcinoma MDA-MB-231 cell, human-derived lung carcinoma H460 cell, human-derived colon carcinoma HCT-116 cell, human-derived lymphoma MV-4-11 cell.

### 3) Experimental Protocol and Results

(1) Results in Human-Derived Hepatic Carcinoma HepG2 Xemograft Model

TABLE 5
Therapeutic effects against human hepatic carcinoma HepG2 xenograft in nude mice

| | Dosage | Administering | Animal Number | Tumor Weight(g) | Inhibition |
|---|---|---|---|---|---|
| Group | (mg/kg) | method | Beginning/End | Mean ± SE | Rate (%) |
| Solvent Control | Solvent | iv, QOD × 14 d | 10/10 | 0.7656 ± 0.0300 | — |
| rapamycin | 10 | iv, QW × 14 d | 10/10 | 0.4806 ± 0.0269** | 37.23% |
| Vinorelbine | 8 | iv, QW × 14 d | 10/10 | 0.4719 ± 0.0660** | 38.36% |
| II1E-01•HCl | 2 | iv, QOD × 14 d | 10/10 | 0.3177 ± 0.0410** | 58.50% |
| II1E-01•HCl | 4 | iv, QOD × 14 d | 10/10 | 0.0938 ± 0.0121** | 87.75% |
| II1E-01•HCl | 6 | iv, QOD × 14 d | 10/10 | 0.0262 ± 0.0054** | 96.58% |

Remarks: comparing with the solvent control group,
*P < 0.05,
**P < 0.01.

HepG2 cells were in vitro cultured to be proliferated. The cells in the log phase of growth were collected and resuspended in DMEM serum-free medium, and then implanted subcutaneously into the axilla of the right forelimb of nude mice. After 11 days, the tumor size reached about 250 mm³. The mice bearing the tumor were grouped according to the tumor size into 6 groups, including the group of solvent control, groups of positive controls (Rapamycin 10 mg/kg and Vinorelbine 8 mg/kg), and groups of low (2 mg/kg), middle (4 mg/kg) and high (6 mg/kg) dosages of the testing compound II1E-01.HCl. Each group was administered intravenously at tail. In the solvent control group, the solvent control solution was administered intravenously once every other day (QOD). In the low, middle and high dosage groups of II1E-01.HCl, II1E-01.HCl was administered intravenously once every other day (QOD). In the positive control groups, Rapamycin 10 mg/kg and Vinorelbine 8 mg/kg were administered once a week (QW). The animals were given the drugs for two weeks. In the two week time period, the growth of tumor and the body weight of the mice were observed.

Two weeks after administering (the 25^{th} day after the implantation), the tumor weight and inhibition rate were calculated, and results are listed in Table 5. As shown in Table 5, the positive control Rapamycin 10 mg/kg and Vinorelbine 8 mg/kg groups have inhibition rates of 37.23% and 38.36% respectively, while the 2 mg/kg, 4 mg/kg and 6 mg/kg groups of the testing compound II1E-01.HCl have inhibition rates of 58.50%, 87.75% and 96.58% respectively. The tumor weights of the positive control groups and the testing groups were significantly different (P<0.01) from that of the solvent control group.

(2) Results in Human-Derived Breast Carcinoma MDA-MB-231 Xemograft Model

TABLE 6
Therapeutic effects against human-derived MDA-MB-231 xenograft in nude mice

| | Dosage | Administering | Animal Number | Tumor Weight(g) | Inhibition |
|---|---|---|---|---|---|
| Group | (mg/kg) | method | Beginning/End | Mean ± SE | Rate (%) |
| Solvent Control | Blank Solvent | iv, QOD × 7 d | 10/10 | 3.1160 ± 0.2051 | — |
| II1E-01•HCl | 2 | iv, QOD × 7 d | 10/10 | 2.6063 ± 0.2731 | 16.36% |
| II1E-01•HCl | 4 | iv, QOD × 7 d | 10/10 | 2.0569 ± 0.1340** | 33.99% |
| II1E-01•HCl | 6 | iv, QOD × 7 d | 10/10 | 1.4887 ± 0.1763** | 52.23% |

Remarks: comparing with the solvent control group,
*P < 0.05,
**P < 0.01.

19 days after MDA-MB-231 cells were implanted, the tumor size reached about 617 mm³. The mice bearing the tumor were grouped and administered. The groups of II1E-01.HCl were administered once every other day (QOD), and the solvent control group was administered with the solvent control solution. After completion of the experiment, the tumor weight and the inhibition rate were calculated, and the results are summarized in Table 6. The high and middle dosage groups have significant inhibitory activities against the growth of MDA-MB-231 xenograft, and the effect depends on the amount obviously.

The tumor in this experiment had been allowed to grow to reach a relative bigger size before drug administration (the tumor size before drug administration was about 600 mm³ while generally the tumor size was only 200-300 mm³ before drug administration), which corresponds to a middle- or advanced-stage cancer. The compound II1E-01.HCl at the middle and high dosage still showed significant growth inhibition activities against the MDA-MB-231 xenograft nude model. The results suggested that II1E-01.HCl might be clinically useful in treating middle- or advanced-stage cancer.

(3) Results in Human-Derived Lung Carcinoma H460 Xemograft Model

TABLE 7
Therapeutic effects against human-derived lung cancer H460 xenograft in nude mice

| | Dosage | Administering | Animal Number | Tumor Weight(g) | Inhibition |
|---|---|---|---|---|---|
| Group | (mg/kg) | method | Beginning/End | Mean ± SE | Rate (%) |
| Solvent Control | Solvent | iv, QOD × 21 d | 10/10 | 1.3468 ± 0.1514 | — |
| II1E-01•HCl | 4 | iv, QOD × 21 d | 10/10 | 0.5125 ± 0.0307** | 61.94% |
| II1E-01•HCl | 6 | iv, QOD × 21 d | 10/10 | 0.3246 ± 0.0692** | 75.90% |

Remarks: comparing with the solvent control group,
**P < 0.01.

NCI-H460 xenograft model was established by tumor implantation. 19 days after the implantation, the tumor size reached about 210 mm³. The mice bearing the tumor were grouped by randomized block method according to the tumor size into 3 groups, which contain 10 mice in each group and include the group of solvent control, and groups of middle (4 mg/kg) and high (6 mg/kg) dosages of the testing compound II1E-01.HCl. In the solvent control group, 5% glucose solution was administered intravenously at tail once every other day (QOD). The testing compound was administered intravenously at tail once every other day (QOD). The animals were given the drugs for three weeks, during which the growth of tumor and the body weight of the mice were observed.

After continuously administering for 21 days (the 40^{th} day after the implantation), the tumor weight and inhibition rate were calculated, and results are listed in Table 7. Both the high (6 mg/kg QOD) and low (4 mg/kg QOD) dosage groups of II1E-01.HCl showed significant antitumor activities with inhibition rates of 75.90% (P<0.01) and 61.94% (P<0.01) respectively.

(4) Results in Human-Derived Colon Carcinoma HCT-116 Xemograft Model

TABLE 8
Therapeutic effects against human-derived colon
carcinoma HCT-116 xenograft in nude mice

| | Dosage | Administering | Animal Number | Tumor Weight(g) | Inhibition |
|---|---|---|---|---|---|
| Group | (mg/kg) | method | Beginning/End | Mean ± SE | Rate (%) |
| Solvent Control | Solvent | iv, QOD × 14 d | 10/10 | 1.2691 ± 0.0966 | — |
| II1E-01•HCl | 4 | iv, QOD × 14 d | 10/10 | 0.8017 ± 0.0480** | 36.82% |
| II1E-01•HCl | 6 | iv, QOD × 14 d | 10/10 | 0.4147 ± 0.0212** | 67.33% |

Remarks: comparing with the solvent control group,
**P < 0.01.

HCT116 cells were in vitro cultured to be proliferated. The cells in the log phase of growth were collected, resuspended in RPMI-1640 serum-free medium, and then implanted subcutaneously into the axilla of the right forelimb of nude mice. After 14 days, the tumor size reached about 470 mm³. The mice bearing the tumor were grouped by randomized block method according to the tumor size into 3 groups, including the group of solvent control and groups of 4 mg/kg and 6 mg/kg dosages of the testing compound II1E-01.HCl. In the solvent control group, glucose injection was administered intravenously at tail once every other day (QOD). The testing compound II1E-01.HCl was administered intravenously (iv) at tail once every other day (QOD). The animals were given the drugs for 14 days, during which, the growth of tumor and the body weight of the mice were observed.

After continuously administering for 14 days (the 28^{th} day after the implantation), the tumor weight and inhibition rate were calculated, and results are listed in Table 8. The testing compound II1E-01.HCl had inhibition rates of 36.82% and 67.33% at 4 mg/kg QOD and 6 mg/kg QOD dosages respectively.

(5) Results in Human-Derived Lymphoma MV-4-11 Xemograft Model

TABLE 9
Therapeutic effects against human-derived leukaemia MV-4-11 xenograft in nude mice

| | Dosage | Administering | Animal Number | Tumor Weight(g) | Inhibition |
|---|---|---|---|---|---|
| Group | (mg/kg) | method | Beginning/End | Mean ± SE | Rate (%) |
| Solvent Control | Solvent | ig, QD × 21 d | 6/6 | 2.8348 ± 0.3005 | — |
| Sunitinib | 40 | ig, QD × 21 d | 6/6 | 0.0000 ± 0.0000** | 100.00% |
| II1E-01•HCl | 4 | iv, QOD × 21 d | 6/6 | 0.0000 ± 0.0000** | 100.00% |

Remarks: comparing with the solvent control group,
**P < 0.01.

MV-4-11 cells were in vitro cultured to be proliferated. The cells in the log phase of growth were collected and resuspended in IMDM serum-free medium, and then implanted subcutaneously into the axilla of the right forelimb of nude mice. After 37 days, the tumor size reached about 270 mm³. The mice bearing the tumor were grouped by randomized block method according to the tumor size into 3 groups, which contain 6 mice in each group and include the group of solvent control, group of positive control (Sunitinib 40 mg/kg), and group of the testing compound II1E-01.HCl (4 mg/kg QOD). In the positive control group, sunitinib was administered intragastrically once daily (QD). In the group of II1E-01.HCl, II1E-01.HCl was administered intravenously at tail once every other day (QOD). The animals were given the drugs for 21 days, during which the growth of tumor and the body weight of the mice were observed.

After continuously administering for 21 days (the 58^{th} day after the implantation), the tumor weight and inhibition rate were calculated, and results are listed in Table 9. The positive control Sunitinib showed significant antitumor activity at the dosage of 40 mg/kg, and the tumor completely disappeared at 10^{th} day after administration, and the inhibition rate was 100.00% (P<0.01) at the end of the experiment. In the group of the testing compound II1E-01.HCl (4 mg/kg QOD), the tumor also completely disappeared at 14^{th} day after administration, and the inhibition rate was 100.00% (P<0.01) at the end of the experiment.

1. Bufadienolide derivatives represented by formula I, or the pharmaceutically acceptable salts thereof, 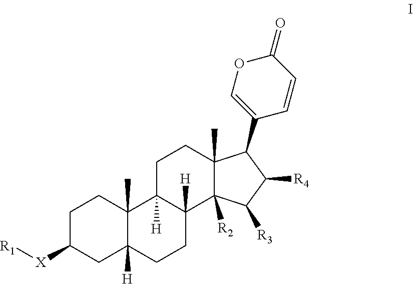
wherein,
X is O or NH;
R₁ is a group selected from any one of the following groups: 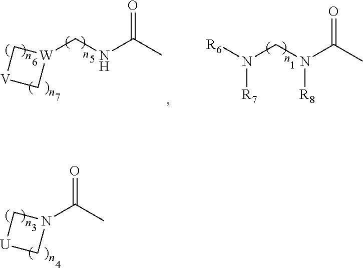
wherein,
n₅ is 0, 1, 2 or 3;
n₆ is 0, 1, 2, 3 or 4;
n₇ is 0, 1, 2, 3 or 4; and n₆ and n₇ are not 0 simultaneously;
W is CH;
V is R₁₅—N;
R₁₅ is H, C₁-C₆ alkyl, —C(═O)R₁₁, —SO₂—R₁₂ or an amino acid residue;
n₁ is 1, 2 or 3;
Y is N;
R₅ is H or C₁-C₆ alkyl;
R₆ and R₇ are each independently H or C₁-C₆ alkyl;
n₃ is 0, 1, 2 or 3;
n₄ is 0, 1, 2, or 3; and n₃ and n₄ are not 0 simultaneously;
U is R₁₃—N or R₁₄—CH;
R₁₃ is H, C₁-C₆ alkyl, —C(═O)R₁₁, —SO₂—R₁₂ or an amino acid residue;
R₁₄ is H, C₁-C₆ alkyl, hydroxyl, C₃-C₇ cycloalkyl, benzyl, aryl, NH₂, amino substituted with C₁-C₄ alkyl or hydroxyl C₁-C₄ alkyl, C₁-C₆ alkyloxy or 5-7 membered heteroaryl;
R₁₁ and R₁₂ are each independently H, C₁-C₆ alkyl, C₃-C₇ cycloalkyl, C₁-C₆ alkyloxycarbonyl, benzyl, aryl, NH₂, amino substituted with C₁-C₆ alkyloxycarbonyl C₁-C₄ alkyl or C₃-C₇ cycloalkyl, amino substituted with benzyl or phenyl, C₁-C₆ alkyloxy or 5-7 membered heteroaryl;
the aryl may be phenyl, naphthyl or biphenyl, or phenyl substituted with 1 to 4 substituents selected from the group consisting of halo, C₁-C₆ alkyl, CN, NO₂, NH₂, hydroxyl, hydroxyl C₁-C₄ alkyl, C₁-C₄ haloalkyl, carboxy, C₁-C₄ alkyloxy, C₁-C₄ haloalkyloxy, mercapto and C₁-C₄ alkyloxycarbonyl;
R₂ is —OH;
R₃ is —H;
R₄ is —H;
with the provision that the above compounds exclude the following compound:

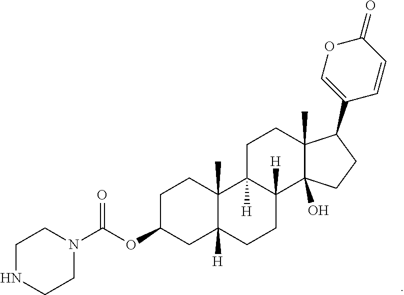

2. The bufadienolide derivatives or the pharmaceutically acceptable salts thereof according to claim 1, wherein
R₁₅ is H or C₁-C₄ alkyl;
n₁ is 2 or 3;
R₅ is H or C₁-C₄ alkyl;
R₆ and R₇ are each independently H or C₁-C₄ alkyl;
R₁₃ is H, C₁-C₄ alkyl, —C(═O)R₁₁ or —SO₂—R₁₂;
R₁₁ and R₁₂ are each independently H, C₁-C₄ alkyl, C₁-C₄ alkyloxycarbonyl, benzyl, aryl, NH₂, amino substituted with C₁-C₄ alkyloxycarbonyl C₁-C₂ alkyl or C₅-C₇ cycloalkyl, amino substituted with benzyl or phenyl, C₁-C₄ alkyloxy or pyridyl;
R₁₄ is H, C₁-C₄ alkyl, hydroxyl, C₃-C₇ cycloalkyl, NH₂, amino substituted with C₁-C₄ alkyl or hydroxyl C₁-C₄ alkyl, or C₁-C₄ alkyloxy;
the aryl may be phenyl, or phenyl substituted with one substituent selected from the group consisting of C₁-C₄ alkyl, NO₂, NH₂, hydroxyl, hydroxyl C₁-C₄ alkyl, C₁-C₄ haloalkyl, carboxy, C₁-C₄ alkyloxy and C₁-C₄ alkyloxycarbonyl;
with the provision that the above compounds exclude the following compound:

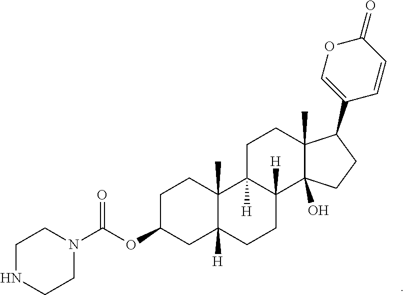

3. The bufadienolide derivatives or the pharmaceutically acceptable salts thereof according to claim 2, wherein
R₁₅ is H, methyl or ethyl;
R₅ is H, methyl or ethyl;
R₆ and R₇ are each independently H, methyl or ethyl;
R₁₃ is H, methyl, ethyl, —C(═O)R₁₁ or —SO₂—R₁₂;
R₁₁ and R₁₂ are each independently H, methyl, ethyl, methoxycarbonyl, ethoxycarbonyl, benzyl, phenyl, phenyl substituted with methyl, NO₂ or methoxycarbonyl, NH₂, amino substituted with ethoxycarboxylethyl, cyclohexyl or benzyl, methoxy, ethoxy or pyridyl;
R₁₄ is H, methyl, hydroxyl, hydroxylethylamino or dimethylamino;
with the provision that the above compounds exclude the following compound:

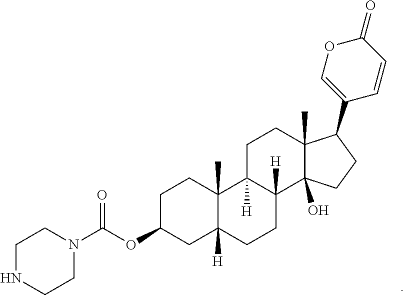

4. The bufadienolide derivatives or the pharmaceutically acceptable salts thereof according to claim 1, wherein
X is O;
R₁ is 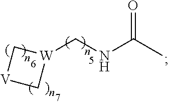
n₅ is 0, 1 or 2;
n₆ is 0, 1, 2, 3 or 4;
n₇ is 0, 1, 2, 3 or 4; and n₆ and n₇ are not 0 simultaneously;
W is CH;
V is R₁₅—N;
R₁₅ is H or C₁-C₆ alkyl.

5. A bufadienolide derivatives and the pharmaceutically acceptable salts thereof, the bufadienolide derivatives being one selected from the group consisting of

| | | | | | I |
|---|---|---|---|---|---|
| 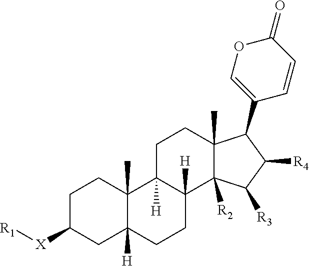 | | | | | |
| No. | X | R₁ | R₂ | R₃ | R₄ |
| II1B-01 | O | 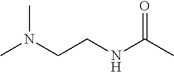 | OH | H | H |
| II1B-02 | O | 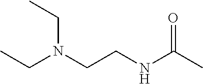 | OH | H | H |
| II1B-03 | O | 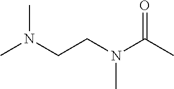 | OH | H | H |
| II1B-04 | O | 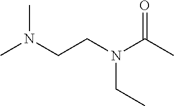 | OH | H | H |
| II1B-05 | O | 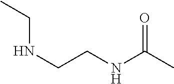 | OH | H | H |
| II1B-06 | O | 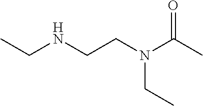 | OH | H | H |
| II1B-07 | O | 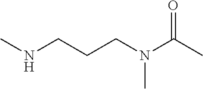 | OH | H | H |
| II1B-08 | O | 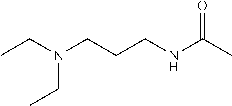 | OH | H | H |
| II1B-09 | O | 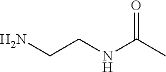 | OH | H | H |
| II1B-10 | O | 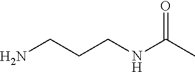 | OH | H | H |
| II1D-01 | O | 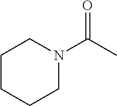 | OH | H | H |
| II1D-03 | O | 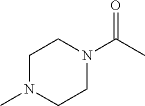 | OH | H | H |
| II1D-04 | O | 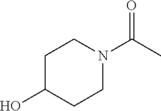 | OH | H | H |
| II1D-06 | O | 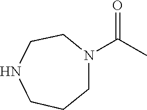 | OH | H | H |
| II1D-07 | O | 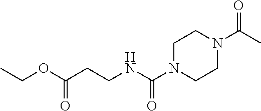 | OH | H | H |
| II1D-08 | O | 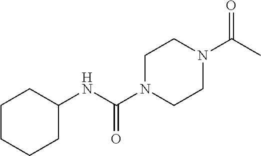 | OH | H | H |
| II1D-09 | O | 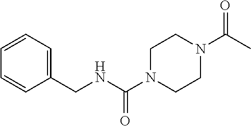 | OH | H | H |
| II1D-10 | O | 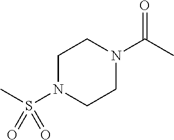 | OH | H | H |
| II1D-11 | O | 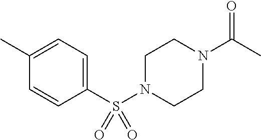 | OH | H | H |
| II1D-12 | O | 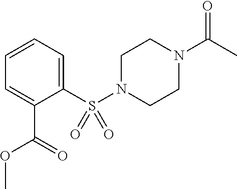 | OH | H | H |
| II1D-13 | O | 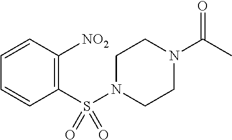 | OH | H | H |
| II1D-14 | O | 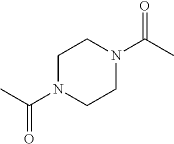 | OH | H | H |
| II1D-15 | O | 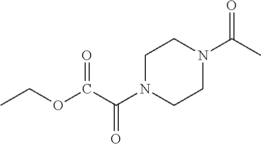 | OH | H | H |
| II1D-16 | O | 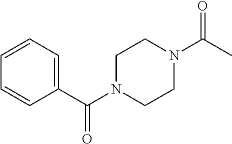 | OH | H | H |
| II1D-19 | O | 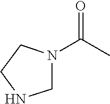 | OH | H | H |
| II1D-20 | O | 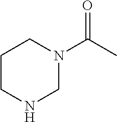 | OH | H | H |
| II1D-22 | O | 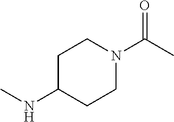 | OH | H | H |
| II1D-23 | O | 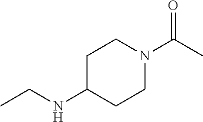 | OH | H | H |
| II1D-24 | O | 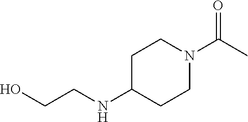 | OH | H | H |
| II1D-25 | O | 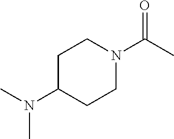 | OH | H | H |
| II1E-01 | O | 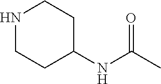 | OH | H | H |
| II1E-02 | O | 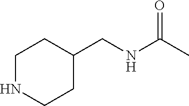 | OH | H | H |
| II1E-03 | O | 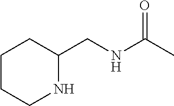 | OH | H | H |
| II1E-04 | O | 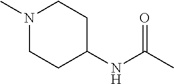 | OH | H | H |
| II1E-05 | O | 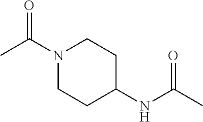 | OH | H | H |
| III1D-02 | NH | 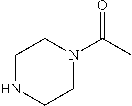 | OH | H | H |
| III1D-03 | NH | 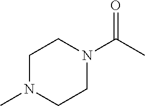 | OH | H | H |
| III1D-04 | NH | 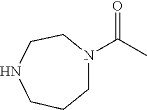 | OH | H | H |
| III1E-01 | NH | 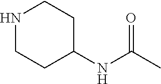 | OH | H | H |
| IIIE-02 | NH | 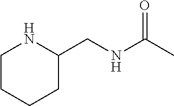 | OH | H | H |

6. A method of preparing a medicament for treating malignancies by using the bufadienolide derivatives and pharmaceutically acceptable salts thereof according to claim 1.

7. The method according to claim 6, wherein the malignancies are selected from liver cancer, lung cancer, breast cancer, stomach cancer, esophageal cancer, colon cancer, leukemia, lymph cancer, prostate cancer, renal cancer, skin cancer, pancreatic cancer, ovarian cancer, brain cancer, bone cancer, and fibrosarcoma.

8. A pharmaceutic composition comprising a therapeutically effective amount of one or more selected from the group consisting of the bufadienolide derivatives and pharmaceutically acceptable salts thereof according to claim 1 as the active component, and optionally, pharmaceutically acceptable vehicles, excipients, adjuvants, and/or auxiliaries.

9. A pharmaceutic composition comprising a therapeutically effective amount of one or more selected from the group consisting of the bufadienolide derivatives and pharmaceutically acceptable salts thereof according to claim 1 as the active component and other pharmaceutically acceptable therapeutic agents, and optionally, pharmaceutically acceptable vehicles, excipients, adjuvants, and/or auxiliaries.

10. A method of treating malignancies comprising administrating a subject having such need with a therapeutically effective amount of one or more selected from the group consisting of the bufadienolide derivatives and pharmaceutically acceptable salts thereof according to claim 1.

11. A method of treating malignancies comprising administrating a subject having such need with the pharmaceutic composition according to claim 8.

12. A method of treating malignancies comprising administrating a subject having such need with the pharmaceutic composition according to claim 9.
